(19) United States
(12) Patent Application Publication
(10) Pub. No.: US 2007/0142386 A1
(43) Pub. Date: Jun. 21, 2007

(54) New 2-substituted, 4-amino-thiazolo[4,5-d] pyrimidines, useful as chemokine receptor antagonists, esp. cx3cr1

(75) Inventors: Gunnar Nordvall, Sodertalje (SE); Tobias Rein, Sodertalje (SE); Daniel Sohn, Sodertalje (SE); Ronald Zemribo, Riga (LV)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca, Sodertalje (SE)

(21) Appl. No.: 10/575,534

(22) PCT Filed: Oct. 5, 2004
(86) PCT No.: PCT/SE04/01421

§ 371 Date: Apr. 7, 2006

(30) Foreign Application Priority Data
Oct. 7, 2003 (SE) 0302666-3
Oct. 7, 2003 (SE) 0302667-1

(51) Int. Cl. A61K 31/525; A61K 31/519; C07D 275/02; C07D 498/02

(52) U.S. Cl. 514/251; 514/260.1; 544/255; 544/259

(57) ABSTRACT

There are disclosed novel compounds of formula (I) wherein A, R¹, R², R³ and X are as defined in the specification, and pharmaceutically acceptable salts thereof, together with processes for their preparation, pharmaceutical compositions comprising them and their use in therapy. The compounds of formula (I) are CX₃CR1 receptor antagonists and are thereby particularly useful in the treatment or prophylaxis of neurodegenerative disorders, demyelinating disease, atherosclerosis and pain.

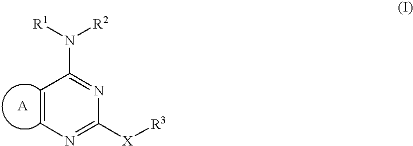

### FIELD OF THE INVENTION

The present invention discloses novel 2-substituted 4-amino-5,6-fused-pyrimidine derivatives together with processes for their preparation, pharmaceutical compositions comprising them and their use in therapy.

### BACKGROUND OF THE INVENTION

Chemokines play an important role in immune and inflammatory responses in various diseases and disorders, including asthma and allergic diseases and inflammatory bowel disease (IBD), as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C) and Cys-Cys (C—C) families. These two groups are distinguished on the basis of a single amino acid insertion between the NH-proximal pair of cysteine residues and sequence similarity.

The C—X—C chemokines include several potent chemoattractants and activators of neutrophils such as interleukin-8 (CXCL8) and neutrophil-activating peptide 2 (CXCL7).

The C—C chemokines include potent chemoattractants of monocytes and lymphocytes but not neutrophils. Examples include human monocyte chemotactic proteins 1-3 (CCL2, CCL7 and CCL8), RANTES (CCL5), eotaxin (CCL11) and the macrophage inflammatory proteins 1α and 1β (CCL3 and CCL4).

There is also a third chemokine family based upon the structural motif Cys-X₃-Cys (C—X₃—C). This C—X₃—C family is distinguished from the C—X—C and C—C families on the basis of having a triple amino acid insertion between the NH-proximal pair of cysteine residues. CX₃CL1 (also known as fractalkine) is a potent chemoattractant and activator of microglia in the central nervous system as well as of monocytes, T cells, NK cells and mast cells.

Studies have demonstrated that the actions of the chemokines are mediated by subfamilies of G protein-coupled receptors. In particular, the actions of CX₃CL1 are mediated by the CX₃CR1 receptor.

WO 01/62758 discloses certain 2-substituted 4-amino-7(8H)-pteridinone derivatives that are useful as antagonists of receptors linked to the C—X—C and C—C chemokine families, particularly as antagonists of the CXCR2 receptor. WO 00/09511 and WO 01/58907 disclose certain 2-substituted 4-amino-thiazolopyrimidine derivatives that are useful as antagonists of receptors linked to the C—X—C and C—C chemokine families, particularly as antagonists of the CXCR2 receptor. WO 01/25242 discloses certain [1,3]thiazolo[4,5-d]pyrimidin-2(3H)-one derivatives that are useful as antagonists of receptors linked to the C—X—C and C—C chemokine families, particularly as antagonists of the CXCR2 receptor.

The present invention relates to a group of compounds that are structurally similar to, but nevertheless generically distinct from, the compounds disclosed in WO 00/09511, WO 01/58907, WO 01/25242 and WO 01/62758. The compounds of the present invention display surprisingly useful properties as antagonists of the CX₃CR1 receptor.

### DISCLOSURE OF THE INVENTION

The present invention provides compounds of formula (I) 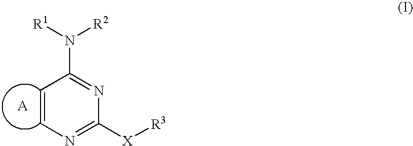
wherein:

A represents a group of formula (a) or (b) or (c):

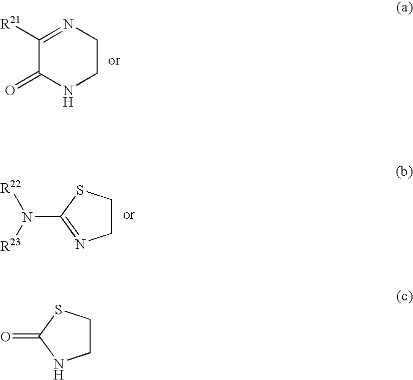

R¹ and R² independently represent H, C1 to 8 alkyl, C2 to 8 alkenyl, C2 to 8 alkynyl or C3 to 7 saturated or partially unsaturated cycloalkyl; the latter four groups being optionally further substituted by one or more groups selected independently from OH, C1 to 6 alkoxy, CH₂OR⁴, NR⁵R⁶, CO₂R⁷ and CONR⁸R⁹;

R³ represents C1 to 6 alkyl, C2 to 6 alkenyl, C2 to 6 alkynyl or C3 to 7 saturated or partially unsaturated cycloalkyl; said alkyl, alkenyl or alkynyl chain optionally including a O, NR¹⁰ or S atom in the chain; said alkyl, alkenyl, alkynyl or cycloalkyl group being optionally substituted by phenyl or a 5 or 6 membered heteroaromatic ring containing 1 to 3 heteroatoms selected independently from O, S and N; said phenyl or heteroaromatic ring being optionally further substituted by one or more groups selected independently from halogen, C1 to 4 alkyl, OH, C1 to 4 alkoxy, CN, CO2R¹¹, NR¹²R¹³, CONR¹⁴R¹⁵, SO₂R¹⁶, NR¹⁷R¹⁸ and SO₂NR¹⁹R²⁰;

X represents O or S(O);

R²¹ represents H, CH₂OR²⁴, CH₂NR²⁴R²⁵, CO₂R²⁴ or CONR²⁴R²⁵;

R²² and R²³ independently represent H, C1 to 6 alkyl, C2 to 6 alkenyl or C3 to 7 saturated or partially unsaturated cycloalkyl; said alkyl, alkenyl or cycloalkyl group being optionally substituted by OR²⁴, NR²⁴R²⁵, CO₂R²⁴ or CONR²⁴R²⁵; or the group —NR²²R²³ together represents a 3 to 7 membered saturated azacyclic ring optionally incorporating one further heteroatom selected from O, S(O)ₙ and NR²⁶; and optionally substituted by OR²⁴, NR²⁴R²⁵, CO₂k²⁴ or CONR²⁴R²⁵;

n represents an integer 0, 1 or 2;

R⁴, R⁵, R⁶, R⁷, R⁸, ⁹, R¹⁰, R¹¹, R¹², R¹³, R¹⁴, R¹⁵, R¹⁶, R¹⁷, R¹⁸, R¹⁹, R²⁰, R²⁴, R²⁵ and R²⁶ independently represent H or C1 to 6 alkyl;

and pharmaceutically acceptable salts thereof.

The compounds of formula (I) may exist in enantiomeric and/or tautomeric forms. It is to be understood that all enantiomers, diastereomers, racemates, tautomers and mixtures thereof are included within the scope of the invention.

Unless otherwise indicated, the term “C1 to 8 alkyl” referred to herein denotes a straight or branched chain alkyl group having from 1 to 8 carbon atoms. Examples of such groups include methyl, ethyl, n-propyl, i-propyl, n-butyl, i-butyl, t-butyl, pentyl and hexyl. The terms “C1 to 6 alkyl” and “C1 to 4 alkyl” are to be interpreted analogously.

Unless otherwise indicated, the term “C2 to 8 alkenyl” referred to herein denotes a straight or branched chain alkyl group having from 2 to 8 carbon atoms and containing one carbon-carbon double bond. The term “C2 to 6 alkenyl” is to be interpreted analogously.

Unless otherwise indicated, the term “C2 to 8 alkynyl” referred to herein denotes a straight or branched chain alkyl group having from 2 to 8 carbon atoms and containing one carbon-carbon triple bond. The term “C2 to 6 alkenyl” is to be interpreted analogously.

Unless otherwise indicated, the term “C3 to 7 saturated or partially unsaturated cycloalkyl” referred to herein denotes a 3 to 7 membered non-aromatic carbocyclic ring optionally incorporating one or more double bonds. Examples include cyclopropyl, cyclopentyl, cyclopentenyl, cyclohexyl and cyclohexenyl.

Unless otherwise indicated, the term “C1 to 6 alkoxy ” referred to herein denotes an oxygen substituent bonded to a straight or branched chain alkyl group having from 1 to 6 carbon atoms. Examples of such groups include methoxy, ethoxy, n-propoxy, i-propoxy, n-butoxy, i-butoxy and s-butoxy. The term “C1 to 4 alkoxy” is to be interpreted analogously.

Unless otherwise indicated, the term “halogen” referred to herein denotes fluorine, chlorine, bromine and iodine.

Examples of a five or six membered heteroaromatic ring containing 1 to 3 heteroatoms independently selected from O, S and N include furan, thiophene, pyrrole, oxazole, oxadiazole, isoxazole, imidazole, thiazole, triazole, thiadiazole, pyridine, pyrimidine and pyrazine.

Examples of a 3 to 7 membered saturated azacyclic ring optionally incorporating one further heteroatom selected from O, S and N include pyrrolidine, piperidine, morpholine and piperazine.

In the definition of R³, the expression “said alkyl, alkenyl or alkynyl chain optionally including a O, NR¹⁰ or S atom in the chain” embraces a straight or branched chain arrangement of 1 to 6 carbon atoms in which, where chemically feasible, the carbon chain is interrupted by, or terminates in, an O, S or NR¹⁰ atom. The definition thus includes, for example, methylene, ethylene, propylene, hexamethylene, ethylethylene, —CH₂CH₂O—CH₂—, —CH₂CH₂O—CH₂—CH₂—, —CH₂CH₂S— and —CH₂CH₂NR¹⁰—.

In one embodiment of the invention, A represents a group of formula (a). That is, compounds of formula (Ia):

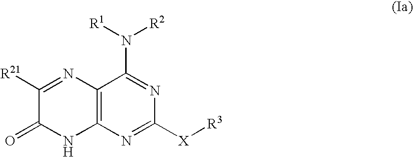

In another embodiment of the invention, A represents a group of formula (b). That is, compounds of formula (Ib):

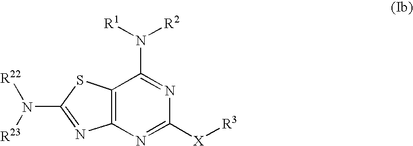

In another embodiment of the invention, A represents a group of formula (c). That is, compounds of formula (Ic):

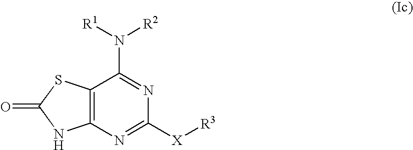

In one embodiment, X represents O. In another embodiment, X represents S(O).

In one embodiment, R²¹ represents H, CO₂R or CO₂NR²⁴R²⁵. In another embodiment, R²¹ represents H.

In one embodiment, R²² and R²³ independently represent H or optionally substituted C1 to 3 alkyl. In another embodiment, R²² and R²³ each represent H.

In one embodiment, R¹ and R² independently represent H, optionally substituted C1 to 8 alkyl or optionally substituted C3 to 7 cycloalkyl.

In another embodiment, R¹ represents H or CH₃. In another embodiment, R¹ represents H.

In another embodiment R² represents optionally substituted C1 to 8 alkyl or optionally substituted C3 to 7 cycloalkyl. In another embodiment, R² represents C1 to 8 alkyl substituted by OH or C3 to 7 cycloalkyl substituted by OH or CH₂OR⁴.

In one embodiment, R³ represents optionally substituted C1 to 6 alkyl that optionally includes an O atom in the chain. In another embodiment, R³ represents C1 to 6 alkyl optionally including an O atom in the chain and substituted by optionally substituted phenyl. In another embodiment, R³ represents C1 to 2 alkyl substituted by phenyl; said phenyl being optionally substituted by halogen, C1 to 6 alkoxy or CN.

In one embodiment, A represents a group of formula (a), X represents O, R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (a), X represents O, R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

In one embodiment, A represents a group of formula (a), X represents S(O), R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (a), X represents S(O), R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

In one embodiment, A represents a group of formula (b), X represents O, R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (b), X represents O, R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

In one embodiment, A represents a group of formula (b), X represents S(O), R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (b), X represents S(O), R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

In one embodiment, A represents a group of formula (c), X represents O, R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (c), X represents O, R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

In one embodiment, A represents a group of formula (c), X represents S(O), R¹ represents H or CH₃; R² represents C1 to 8 alkyl substituted by OH or C3 to 7 cycloalkyl substituted by OH or CH₂OR⁴; and R³ represents C1 to 6 alkyl substituted by optionally substituted phenyl.

In another embodiment, A represents a group of formula (c), X represents S(O), R¹ represents H; R² represents C1 to 8 alkyl substituted by OH or C3 to 7 cycloalkyl substituted by OH or CH₂OR⁴; and R³ represents C1 to 2 alkyl substituted by phenyl; said phenyl being optionally substituted by halogen, C1 to 6 alkoxy or CN.

Particular compounds of formula (I) include:
(2R)-2-{[2-amino-5-(benzyloxy)[1,3]thiazolo[4,5-d]pyrimidin-7-yl]amino}-4-methylpentan-1-ol;
(2R)-2-({2-amino-5-[(3-methoxybenzyl)oxy][1,3]thiazolo[4,5-d]pyrimidin-7-yl}amino)-4-methylpentan-1-ol;
(2R)-2-{[2-amino-5-(2-phenylethoxy)[1,3]thiazolo[4,5-d]pyrimidin-7-yl]amino}-4-methylpentan-1-ol;
(2R)-2-{[2-amino-5-(2-phenoxyethoxy)[1,3]thiazolo[4,5-d]pyrimidin-7-yl]amino}-4-methylpentan-1-ol;
(2R)-2-[{2-amino-5-[(2-methylbenzyl)oxy][1,3]thiazolo[4,5-d]pyrimidin-7-yl}(methyl)amino]-4-methylpentan-1-ol;
(2R)-2-[{2-amino-5-[(4-chlorobenzyl)oxy][1,3]thiazolo[4,5-d]pyrimidin-7-y}(methyl)amino]-4-methylpentan-1-ol;
(2R)-2-[(2-amino-5-[(3-chlorobenzyl)oxy][1,3]thiazolo[4,5-d]pyrimidin-7-yl}(methyl)amino]-4-methylpentan-1-ol;
(2R)-2-[{2-amino-5-[(2-methoxybenzyl)oxy][1,3]thiazolo[4,5-d]pyrimidin-7-yl}(methyl)amino]-4-methylpentan-1-ol;
(2R)-2-[[2-amino-5-(benzyloxy)[1,3]thiazolo[4,5-d]pyrimidin-7-yl](methyl)amino]-4-methylpentan-1-ol;
(2R)-[{2-amino-5-[(4-bromo-2-fluorobenzyl)-(R_{S},S_{S})-sulfinyl][1,3]thiazolo[4,5-d]pyrimidin-7-yl}(methyl)amino]-4-methylpentan-1-ol;
(2R)-2-[(2-amino-5-{[2-(4-bromophenyl)ethyl]-(R_{S},S_{S})-sulfinyl}[1,3]thiazolo[4,5-d]pyrimidin-7-yl)amino]4-methylpentan-1-ol;
(2R)-2-[(2-amino-5-{[2-(2-bromophenyl)ethyl]-(R_{S},S_{S})-sulfinyl}[1,3]thiazol[4,5-d]pyrimidin-7-yl)amino]4-methylpentan-1-ol;
(R)-2-[(2-amino-5-{[2-(2-bromophenyl)ethyl]-(R_{S},S_{S})-sulfinyl}[1,3]thiazolo[4,5-d]pyrimidin-7-yl)(methyl)amino]-4-methylpentan-1-ol;
2-[(2,3-difluorobenzyl)oxy]-4-{[(1R)-1-(hydroxymethyl)-3-methylbutyl]amino}pteridin-7(8H)-one;
4-[(1R)-1-(hydroxymethyl)-3-methylbutyl]amino}-2-[(3-methoxybenzyl)oxy]pteridin-7(8H)-one;
2-[(2-chloro-3-methoxybenzyl)oxy]-4-[(1R)-1-(hydroxymethyl)-3-methylbutyl]amino}pteridin-7(8H)-one;
4-([(1R)-1-(hydroxymethyl)-3-methylbutyl]amino}-2-(2-phenylethoxy)pteridin-7(8H)-one;
4-{[(1R)-1-(hydroxymethyl)-3-methylbutyl]amino}-2-(2-phenoxyethoxy)pteridin-7(8H)-one;
2-[(2-chlorobenzyl)oxy]-4-{[(1R)-1-(hydroxymethyl)-3-methylbutyl]amino}pteridin-7(8H)-one;
2-[(4-chlorobenzyl)oxy]-4-{[(1R)-1-(hydroxymethyl)-3-methylbutyl]amino}pteridin-7(8H)-one;
4-{[(1R)-1-(hydroxymethyl)-3-methylbutyl]amino}-2-[(4-methylbenzyl)oxy]pteridin-7(8H)-one;
4-[(1R)-1-(hydroxymethyl)-3-methylbutyl]amino}-2-[(3-methylbenzyl)oxy]pteridin-7(8H)-one;
2-[(3-chlorobenzyl)oxy]-4-{[(1S,2S)-2-hydroxy-1-(hydroxymethyl)propyl]amino}-7-oxo-7,8-dihydropteridine-6-carboxamide;
2-[(2,3-difluorobenzyl)-(R_{S},S_{S})-sulfinyl]-4-{[(1R)-1-(hydroxymethyl)-3-methylbutyl]amino}pteridin-7(8H)-one;
5-(benzyloxy)-7-{[(1R)-1-(hydroxymethyl)-3-methylbutyl]amino}[1,3]thiazolo[4,5-d]pyrimidin-2(3H)-one;
7-{[(1R)-1-(hydroxymethyl)-3-methylbutyl]amino}-5-[(3-methoxybenzyl)oxy][1,3]thiazolo[4,5-d]pyrimidin-2(3H)-one;
7-{[(1R)-1-(hydroxymethyl)-3-methylbutyl]amino}-5-(2-phenylethoxy)[1,3]thiazolo[4,5-d]pyrimidin-2(3H)-one;
5-(benzyloxy)-7-{[(1R)-1-(hydroxymethyl)butyl]amino}[1,3]thiazolo[4,5-d]pyrimidin-2(3H)-one;
7-{[(1R)-1-(hydroxymethyl)butyl]amino}-5-{[(1S)-1-phenylethyl]oxy)[1,3]thiazolo[4,5-d]pyrimidin-2(3H)-one;
N-(3-{[(7-{[(1R)-1-(hydroxymethyl)butyl]amino}-2-oxo-2,3-dihydro[1,3]thiazolo[4,5-d]pyrimidin-5-yl)oxy]methyl}phenyl)-N-methylmethanesulfonamide;
N-(3-{[(7-{[(1R)-1-(hydroxymethyl)-2-methylpropyl]amino}-2-oxo-2,3-dihydro[1,3]thiazolo[4,5-d]pyrimidin-5-yl)oxy]methyl}phenyl)methanesulfonamide;
5-(benzyloxy)-7-{[1-(hydroxymethyl)cyclopentyl]amino}[1,3]thiazolo[4,5-d]pyrimidin-2(3H)-one;
7-{[1-(hydroxymethyl)cyclopentyl]amino}-5-[(2-methylbenzyl)oxy][1,3]thiazolo[4,5-d]pyrimidin-2(3H)-one;
7-{[1-(hydroxymethyl)cyclopentyl])amino}-5-[(3-methylbenzyl)oxy][1,3]thiazolo[4,5-d]pyrimidin-2(3H)-one;
5-[(2-chlorobenzyl)oxy]-7-{[1-(hydroxymethyl)cyclopentyl)amino}[1,3]thiazolo[4,5-d]pyrimidin-2(3H)-one;
5-[(3-chlorobenzyl)oxy]-7-{[1-(hydroxymethyl)cyclopentyl]amino}[1,3]thiazolo[4,5-d]pyrimidin-2(3H)-one;
5-[(4-chlorobenzyl)oxy]-7-{[1-(hydroxymethyl)cyclopentyl]amino}[1,3]thiazolo[4,5-d]pyrimidin-2(3H)-one;
7-{[1-(hydroxymethyl)cyclopentyl]amino)-5-[(2-methoxybenzyl)oxy][1,3]thiazolo[4,5-d]pyrimidin-2(3H)-one;
7-{[1-(hydroxymethyl)cyclopentyl]amino}-5-[(3-methoxybenzyl)oxy][1,3]thiazolo[4,5-d]pyrimidin-2(3H)-one;
4-{[(7-{[1-(hydroxymethyl)cyclopentyl]amino}-2-oxo-2,3-dihydro[1,3]thiazolo[4,5-d]pyrimidin-5-yl)oxy]methyl}benzonitrile;
(R,S)-7-[[1-(hydroxymethyl)cyclopentyl]amino]-5-(1-phenylethoxy)-thiazolo[4,5-d]pyrimidin-2(3H)-one;
7-{[1-(hydroxymethyl)cyclopentyl]amino}-5-{[(1S)-1-phenylethyl]oxy}[1,3]thiazolo[4,5-d]pyrimidin-2(3H)-one;
5-{[2-(3-chlorophenyl)ethyl]-(R_{S},S_{S})-sulfinyl}-7-{[(1R)-1-(hydroxymethyl)-3-methylbutyl]amino}[1,3]thiazolo[4,5-d]pyrimidin-2(3H)-one;
5-{[2-(2-bromophenyl)ethyl]-(R_{S},S_{S})-sulfinyl}-7-{[(1R)-1-(hydroxymethyl)-3-methylbutyl]amino}[1,3]thiazolo[4,5-d]pyrimidin-2(3H)-one;
5-[(2,3-difluorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one;
5-[benzyl-(R_{S},S_{S})-sulfinyl]-7-{[(1R)-1-(hydroxymethyl)-3-methylbutyl]amino}[1,3]thiazolo[4,5-d]pyrimidin-2(3H)-one;
5-[(2-chlorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one;
5-[(4-chlorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one;
5-[benzyl-(R_{S},S_{S})-sulfinyl]-7-{[(1R)-1-(hydroxymethyl)-2-methylpropyl]amino}[1,3]thiazolo[4,5-d]pyrimidin-2(3H)-one;

and pharmaceutically acceptable salts thereof.

According to the invention, we further provide a process for the preparation of a compound of formula (I), or a pharmaceutically acceptable salt, enantiomer or racemate thereof which comprises:

(a) when X in formula (I) represents O, reaction of a compound of formula (II)

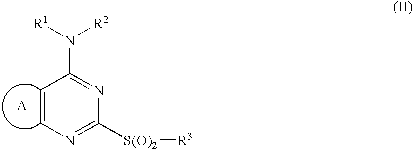

wherein A, R¹, R² and R³ are as defined in formula (I);

with a compound of formula (III)
R³—OH (III)

wherein R³ is as defined in formula (I) and is independent of the R³ group in formula (II); or

(b) when X in formula (I) represents S(O), oxidation of a compound of formula (IV)

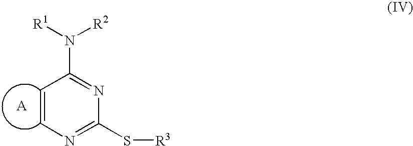

wherein A, R¹, R² and R³ are as defined in formula (I); with one equivalent of an oxidising agent;

and where necessary converting the resultant compound of formula (I), or another salt thereof, into a pharmaceutically acceptable salt thereof; or converting the resultant compound of formula (I) into a further compound of formula (I); and where desired converting the resultant compound of formula (I) into an optical isomer thereof.

In process (a), the reactants (II) and (III) are coupled together in a suitable inert organic solvent such as tetrahydrofuran, benzene, toluene or N-methylpyrrolidine. The reaction is performed in the presence of an added base such as sodium hydride, butyl lithium or lithium diisopropylamide. The reaction is conducted at a suitable temperature, normally between room temperature and the boiling point of the solvent. The reaction is generally continued for a period of about one hour to one week, or until analysis indicates that formation of the required product is complete.

In process (b), the compound is oxidised using one equivalent of a suitable oxidising agent such as those known in the art for the oxidation of sulphides into sulphoxides. A preferred oxidant is oxone. The reaction is generally conducted at ambient temperature and in a suitable solvent such as methanol or aqueous acetonitrile.

Compounds of formula (I) and intermediate compounds thereto may be prepared as such or in protected form. Protecting groups that are suitable for particular functional groups and details of processes for adding and removing such protecting groups are, in general, well known in the art. See, for example, “Protective Groups in Organic Synthesis”, 3rd Edition (1999) by Greene and Wuts.

The present invention includes compounds of formula (I) in the form of salts. Suitable salts include those formed with organic or inorganic acids or organic or inorganic bases. Such salts will normally be pharmaceutically acceptable although salts of non-pharmaceutically acceptable acids or bases may be of utility in the preparation and purification of the compound in question. Thus, preferred acid addition salts include those formed from hydrochloric, hydrobromic, sulphuric, phosphoric, citric, tartaric, lactic, pyruvic, acetic, succinic, fumaric, maleic, methanesulphonic and benzenesulphonic acids. Preferred base addition salts include those in which the cation is sodium, potassium, calcium, aluminium, lithium, magnesium, zinc, choline, ethanolamine or diethylamine.

Salts of compounds of formula (I) may be formed by reacting the free compound, or a salt, enantiomer or racemate thereof, with one or more equivalents of the appropriate acid or base. The reaction may be carried out in a solvent or medium in which the salt is insoluble or in a solvent in which the salt is soluble, for example, water, dioxan, ethanol, tetrahydrofuran or diethyl ether, or a mixture of solvents, which may be removed in vacuo or by freeze drying. The reaction may also be a metathetical process or it may be carried out on an ion exchange resin.

Sulphone derivatives of formula (II) may be prepared by oxidation of the corresponding sulphides of formula (IV) using two or more equivalents of an oxidising agent such as oxone.

In general, compounds of formula (IV) may be prepared using known methods that will be readily apparent to the man skilled in the art. Some such methods are illustrated in Schemes 1 to 5:

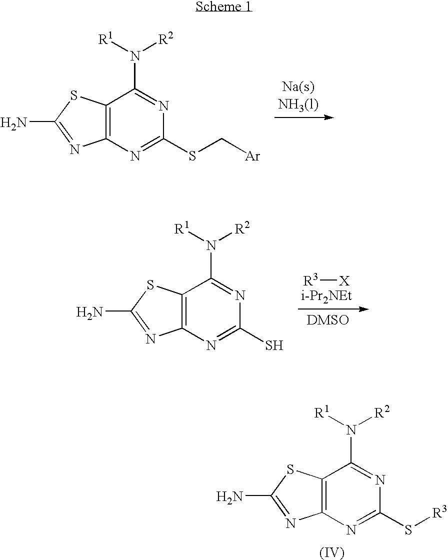

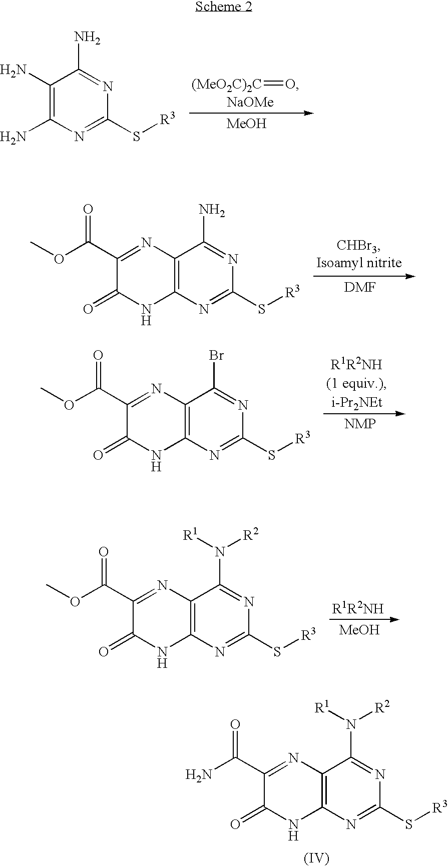

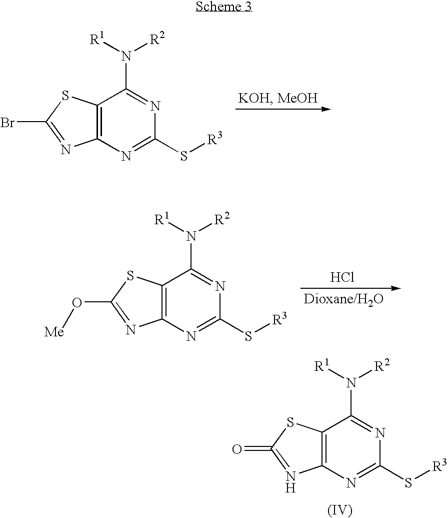

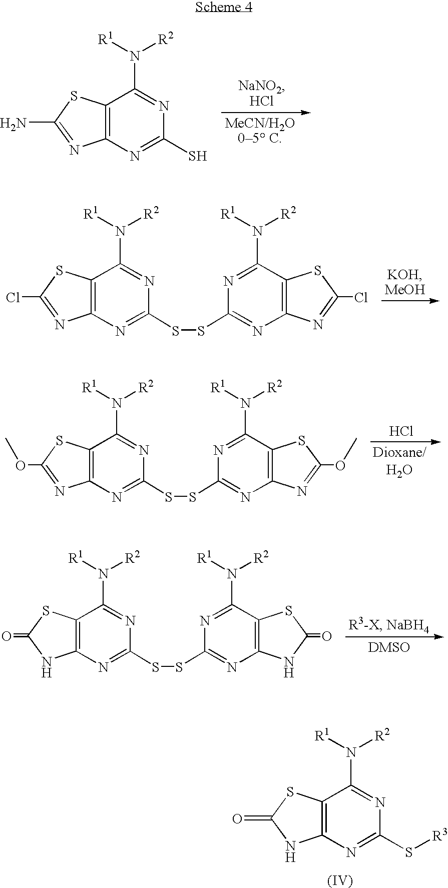

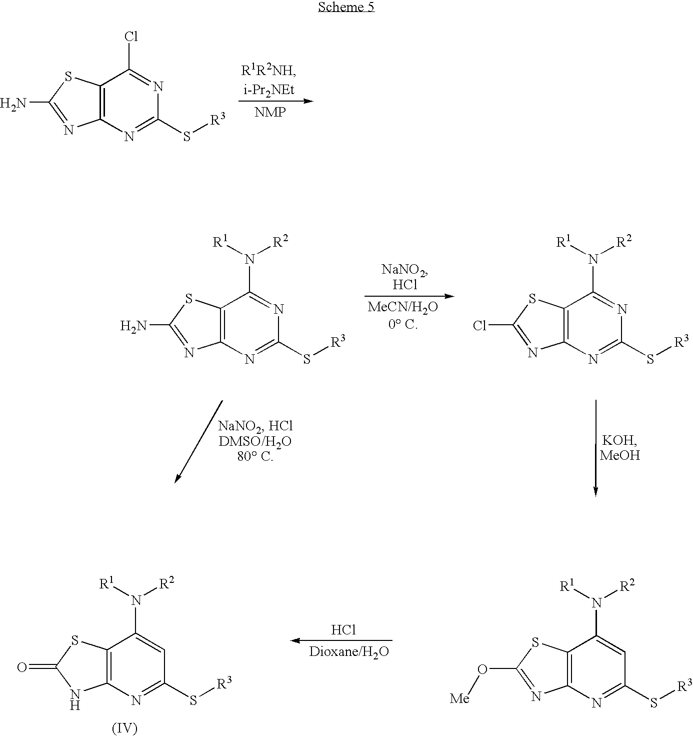

Intermediate compounds may be used as such or in protected form. Protecting groups and details of processes for their removal may be found by reference to the standard text “Protective Groups in Organic Synthesis”, 3rd Edition (1999) by Greene and Wuts.

The compounds of the invention and intermediates thereto may be isolated from their reaction mixtures and, if necessary further purified, by using standard techniques.

The compounds of formula (I) may exist in enantiomeric forms. Therefore, all enantiomers, diastereomers, racemates and mixtures thereof are included with the scope of the invention. The various optical isomers may be isolated by separation of a racemic mixture of the compounds using conventional techniques, for example, fractional crystallisation, or HPLC. Alternatively, the various optical isomers may be prepared directly using optically active starting materials.

Intermediate compounds may also exist in enantiomeric forms and may be used as purified enantiomers, diastereomers, racemates or mixtures.

The compounds of formula (I), and their pharmaceutically acceptable salts are useful because they possess pharmacological activity as antagonists of the CX₃CR1 receptor. In particular, when compared to similar sulphide derivatives disclosed in WO 00/09511, WO 01/58907, WO 01/25242 and WO 01/62758, the ether [formula (I); X═O] and sulphoxide [formula (I); X═S(O)] derivatives of the present invention possess significantly improved solubility profiles.

In one aspect the present invention provides a compound of formula (I) or a pharmaceutically acceptable salt thereof, for use as a medicament.

In another aspect the present invention provides the use of a compound of formula (I) or a pharmaceutically acceptable salt thereof, in the manufacture of a medicament, for the treatment or prophylaxis of diseases or conditions in which antagonism of the CX₃CR1 receptor is beneficial.

In another aspect the present invention provides the use of a compound of formula (I) or a pharmaceutically acceptable salt thereof, in the manufacture of a medicament, for the treatment or prophylaxis of neurodegenerative disorders, demyelinating disease, atherosclerosis or pain.

In another aspect the present invention provides the use of a compound of formula (I) or a pharmaceutically acceptable salt thereof, in the manufacture of a medicament, for the treatment or prophylaxis of multiple sclerosis (MS).

According to the invention, there is also provided a method of treating, or reducing the risk of, diseases or conditions in which antagonism of the CX₃CR1 receptor is beneficial which comprises administering to a person suffering from or at risk of, said disease or condition, a therapeutically effective amount of a compound of formula (I) or a pharmaceutically acceptable salt thereof.

There is also provided a method of treating, or reducing the risk of, neurodegenerative disorders, demyelinating disease, atherosclerosis or pain in a person suffering from or at risk of, said disease or condition, wherein the method comprises administering to the person a therapeutically effective amount of a compound of formula (I) or a pharmaceutically acceptable salt thereof.

There is also provided a method of treating, or reducing the risk of, multiple sclerosis (MS) in a person suffering from or at risk of, said disease or condition, wherein the method comprises administering to the person a therapeutically effective amount of a compound of formula (I) or a pharmaceutically acceptable salt thereof.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of diseases or conditions in which antagonism of the CX₃CR1 receptor is beneficial.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of neurodegenerative disorders, demyelinating disease, atherosclerosis or pain.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of multiple sclerosis.

The compounds of formula (I) and their pharmaceutically acceptable salts are indicated for use in the treatment or prophylaxis of diseases or conditions in which modulation of activity at the CX₃CR1 receptor is desirable. In particular, the compounds are indicated for use in the treatment of neurodegenerative disorders or demyelinating disease in mammals including man. More particularly, the compounds are indicated for use in the treatment of multiple sclerosis. The compounds are also indicated to be useful in the treatment of pain, rheumatoid arthritis, osteoarthritis, stroke, atherosclerosis and pulmonary arterial hypertension.

Conditions that may be specifically mentioned are: neurodegenerative diseases and dementia disorders, for example, Alzheimer's disease, amyotrophic lateral sclerosis and other motor neuron diseases, Creutzfeldt-Jacob's disease and other prion diseases, HIV encephalopathy, Huntington's disease, frontotemporal dementia, Lewy body dementia and vascular dementia; polyneuropathies, for example, Gulllain-Barré syndrome, chronic inflammatory demyelinating polyradiculoneuropathy, multifocal motor neuropathy and plexopathies; CNS demyelination, for example, acute disseminated/haemorrhagic encephalomyelitis and subacute sclerosing panencephalitis; neuromuscular disorders, for example, myasthenia gravis and Lambert-Eaton syndrome; spinal disorders, for example, tropical spastic paraparesis and stiff-man syndrome; paraneoplastic syndromes, for example, cerebellar degeneration and encephalomyelitis; CNS trauma; and migraine.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The compounds of the invention are also indicated for use in the treatment of inflammatory bowel disease (IBD), for example, Crohn's disease and ulcerative colitis, by inducing remission and/or maimtaining remission of IBD.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formula (I) and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formula (I), or a pharmaceutically acceptable salt thereof.

There is also provided a process for the preparation of such a pharmaceutical composition that comprises mixing the ingredients.

The invention is illustrated, but in no way limited, by the following examples:

General Procedures

Nuclear magnetic resonance (NMR) spectra were recorded on a Varian Gemini 7 Tesla 300 MHz instrument, or a Bruker Avance 400 MHz instrument using the solvent indicated. Chemical shifts are given in ppm down- and upfield from tetramethylsilane (TMS). Resonance multiplicities are denoted s, d, t, m, br and app for singlet, doublet, triplet, multiplet, broad and apparent, respectively. Mass spectra (MS) were recorded on a Finnigan SSQ7000 TSP or a Finnigan SSQ710 DI/EI instrument, or on a single quadropole mass spectrometer, ZMD (Waters), using an electrospray ion source operated in a positive mode. The ion spray voltage was +3 kV and the mass spectrometer was scanned from m/z 100-900 with a scan time of 0.85 s. LC-MS was performed with a Waters 2790 LC-system equipped with a Waters Xterra™ MS C₈ (2.5 μm×30 mm) column, a Waters 996 photodiode array detector and a Micromass ZMD. High pressure liquid chromatography (HPLC) assays were performed using a Hewlett Packard 1100 Series HPLC system equipped with a Zorbax SB-C₈ (4.6 mm×15 cm) column. Preparative high pressure liquid chromatography (prep HPLC) separations were performed on an automated Gilson (model 170) using an Xterra C₁₈ (19 mm×30 cm) column, and using a gradient of A (water 95%, containing NH₄OAc (0.01 M), and 5% CH₃CN) and B (CH₃CN) as eluent. Column chromatography was performed using silica gel 60 (230-400 mesh ASTM, Merck) and thin layer chromatography (TLC) was performed on TLC precoated plates, silica gel 60 F₂₅₄ (Merck).

### EXAMPLE 1

### (2R)-2-{[2-Amino-5-(benzyloxy)[1,3]thiazolo[4,5-d]pyrimidin-7-yl]amino}-4-methylpentan-1-ol

### (a) (2R)-2-{[2-Amino-5-(benzylsulfonyl)[1,3]thiazolo[4,5-d]pyrimidin-7-yl]amino}-4-methylpentan-1-ol

(2R)-2-{[2-Amino-5-(benzylthio)[1,3]thiazolo[4,5-d]pyrimidin-7-yl]amino}-4-methylpentan-1-ol (WO 00/09511) (1.0 g, 2.56 mmol) was dissolved in CH₃CN (120 mL) and water (80 mL). Potassium peroxymonosulfate (Oxone, 3.38 g, 5.50 mmol) was added and the resulting slurry was stirred at RT for 16 h. Na₂S₂O₃ solution was added and the CH₃CN was evaporated. The residue was poured onto ice and the precipitate was collected by filtration, washed with water and dried in vacuo at 40° C. overnight resulting in 920 mg (85%) of the title compound as an off-white solid.

¹H NMR (DMSO-d₆) δ 8.40-8.19 (br s, 2H), 7.40-7.26 (m, 5H), 6.83 (d, 1H), 4.84 (d, 1H), 4.77 (d, 1H), 4.40 (br s, 1H), 3.62-3.43 (m, 3H), 1.63-1.39 (m, 3H), 0.91 (d, 3H), 0.84 (d, 3H);

MS (ESI⁺) m/z 422 [M+H]⁺.

### (b) (2R)-2-{[2-Amino-5-(benzyloxy)[1,3]thiazolo[4.5-d]pyrimidin-7-yl]amino}-4-methylpentan-1-ol

Solid NaH (17 mg, 0.7 mmol; 7 eq.) was added to a stirred solution of benzyl alcohol (76 mg, 0.7 mmol; 7 eq.) in dry benzene (5 mL) at 0° C. The solution was allowed to reach RT over 15 min. The product of step (a) (42 mg, 0.1 mmol; 1 eq.) was added as a solid, and the mixture was heated to reflux for 1 h. After cooling to RT, the reaction was quenched by the addition of saturated NH₄Cl solution (1 mL). The mixture was partitioned between THF (10 mL) and water (10 mL). The organic phase was separated, dried over Na₂SO₄ and evaporated in vacuo. The oily residue was purified by preparative HPLC to give the title compound as an off-white solid (4.8 mg, 13%).

¹H NMR (DMSO-d₆) δ 8.04 (br s, 2H), 7.41-7.25 (m, 5H), 6.89 (d, 1H), 5.26 (s, 2H), 4.74-4.60 (m, 2H), 3.50-3.33 (m, 2H), 1.63-1.39 (m, 2H), 1.27 (m, 1H), 0.90 (d, 3H), 0.83 (d, 3H);

MS (ESI⁺) m/z 374 [M+H]⁺.

The compounds of Examples 2 to 4 were prepared using the general method of Example 1, step (b), but replacing benzyl alcohol with the appropriate alcohol.

### EXAMPLE 2

### (2R)-2-({2-Amino-5-[(3-methoxybenzyl)oxy][1,3]thiazolo[4,5-d]pyrimidin-7-yl}amino)-4-methylpentan-1-ol

Off-white solid (4.4 mg, 11% yield).

¹H NMR (DMSO-d₆) δ 8.11 (br s, 2H), 7.39-7.33 (2H), 7.10 (d, 1H), 6.95 (s, 1H) 6.80 (d, 1H), 5.26 (s, 2H), 4.77-4.57 (m, 2H), 3.48-3.39 (m, 2H), 3.33 (s, 3H), 1.55-1.37 (m, 2H), 1.26 (m, 1H), 0.89 (d, 3H), 0.83 (d, 3H);

MS (ESI⁺) m/z 404 [M+H]⁺.

### EXAMPLE 3

### (2R)-2-{[2-Amino-5-(2-phenylethoxy)[1,3]thiazolo[4,5-d]pyrimidin-7-yl]amino}-4-methylpentan-1-ol

Off-white solid (6.2 mg, 16% yield).

¹H NMR (DMSO-d₆) δ 8.10 (br s, 2H), 7.35-7.22 (m, 5H), 6.83 (d, 1H), 4.83 (t, 2H), 4.77-4.50 (m, 2H), 3.58-3.44 (m, 2H), 3.23 (t, 2H), 1.50-1.39 (m, 2H), 1.29 (m, 1H), 0.89 (d, 3H), 0.84 (d, 3H);

MS (ESI⁺) m/z 388 [M+H]⁺.

### EXAMPLE 4

### (2R)-2-{[2-Amino-5-(2-phenoxyethoxy)[1,3]thiazolo[4,5-d]pyrimidin-7-yl]amino}-4-methylpentan-1-ol

Clear film (12% yield).

¹H NMR (CD₃OD) δ 7.27-7.15 (m, 2H), 6.95-6.82 (m, 3H), 4.85 (protons in the water peak, 4H), 4.78-4.63 (m, 2H), 4.58-4.21 (m, 1H), 4.23-4.12 (m, 2H) 3.53-3.35 (m, 2H), 1.81-1.68 (m, 1H), 1.68-1.24 (m, 2H), 0.98-0.83 (m, 6H);

MS (ESI⁺) m/z 404 [M+H]⁺.

### EXAMPLE 5

### (2R)-2-{[2-Amino-5-[(2-methylbenzyl)oxy][1,3]thiazolo[4,5-d]pyrimidin-7-yl}(methyl)amino]-4-methylpentan-1-ol

### (a) (2R)-2-[[2-Amino-5-(benzylthio)[1,3]thiazolo[4,5-d]pyrimidin-7-yl](methyl)amino]-4-methylpentan-1-ol

5-(Benzylthio)-7-chloro[1,3]thiazolo[4,5-d]pyrimidin-2-amine (WO 00/09511) (1.5 g, 4.86 mmol), N-ethyl-N,N-diisopropylamine(DIPEA) (691 mg, 5.35 mmol) and (R)-N-methylleucinol (Aitali, M.; Allaoud, S.; Karim, A.; Meliet, C.; Mortreux, A. Tetrahedron: Asymmetry 2000, 11, 1367-1374) (956 mg, 7.29 mmol) were mixed in 1-methyl-2-pyrrolidinone (N) (7.5 mL). The resulting solution was stirred at 110° C. under a nitrogen atmosphere for 2 days. After cooling to RT the reaction mixture was poured onto ice. The resulting yellow precipitate was collected by filtration, washed with water and dried in vacuo. The crude product was purified by column chromatography on silica (CH₂Cl₂:EtOAc 50:50 to 0:100) to give 1.42 g (72% yield) of the title compound as a pale yellow solid.

¹H NMR (DMSO-d₆) δ 7.97 (br s, 2H), 7.40 (m, 2H), 7.28 (m, 2H), 7.21 (m, 1H), 4.73 (dd, 1H), 4.64 (br s, 1H), 4.32 (br s, 2H), 3.52-3.37 (m, 2H), 3.00 (s, 3H), 1.55-1.35 (m, 2H), 1.31-1.22 (m, 1H), 0.88 (d, 3H), 0.80 (d, 3H);

MS (ESI⁺) m/z 404 [M+H]⁺.

### (b) (2R)-2-[[2-Amino-5-(benzylsulfonyl)[1,3]thiazolo[4,5-d]pyrimidin-7-yl](methyl)amino]4-methylpentan-1-ol

Oxidation of the product from step (a) according to the procedure described in Example 1, step (a), gave the title compound as an off-white solid in 80% yield.

¹H NMR (DMSO-d₆) δ 8.32 (br s, 2H), 7.41-7.29 (m, 5H), 4.87 (d, 1H), 4.78 (d, 1H) overlapping with 4.72 (br s, 1H), 3.60-3.41 (m, 2H), 3.11 (s, 3H), 1.60-1.39 (m, 2H), 1.35-1.25 (m, 1H), 0.90 (d, 3H), 0.85 (d, 3H);

MS (ESI⁺) m/z 436 [M+H]⁺.

### (c) (2R)-2-[{2-Amino-5-[(2-methylbenzyl)oxy][1,3]thiazolo[4,5-d]pyrimidin-7-yl}(methyl)amino]-4-methylpentan-1-ol

2-Methylbenzyl alcohol (141 mg, 1.15 mmol) was dissolved in dry THF (200 μl) under a nitrogen atmosphere and the solution was cooled to −20° C. n-Butyl lithium (1.6M in hexane, 360 μl, 0.58 mmol) was added dropwise and the resulting solution was stirred for 10 min. The product of step (b) (50 mg, 0.12 mmol) was added and the reaction mixture was heated to 50° C. for 3 h. After cooling to RT, aqueous NH₄Cl followed by EtOAc were added and the phases were separated. The water phase was extracted three times with EtOAc and the combined organic extracts were dried over anhydrous MgSO₄, filtered and concentrated. Purification by preparative HPLC (eluent CH₃CN:0.1M NH₄OAc 30:70 to 70:30) gave the title compound as an off-white solid (3 mg, 6% yield).

¹H NMR (DMSO-d₆) δ 7.89 (br s, 2H), 7.37 (d, 1H), 7.25-7.14 (m, 3H), 5.27 (s, 2H), 4.76-4.61 (br s, 1H) overlapping with δ 4.72 (br s, 1H), 3.52-3.37 (m, 2H), 3.01 (s, 3H), 2.32 (s, 3H), 1.56-1.37 (m, 2H), 1.33-1.23 (m, 1H), 0.87 (d, 3H), 0.82 (d, 3H);

MS (ESI⁺) m/z 402 [M+H]⁺.

The compounds of Examples 6 to 9 were prepared using the general method of Example 5, step (c), but replacing benzyl alcohol with the appropriate alcohol.

### EXAMPLE 6

### (2R)-2-[{2-Amino-5-[(4-chlorobenzyl)oxy][1,3]thiazolo[4,5-d]pyrimidin-7-yl}(methyl)amino]-4-methylpentan-1-ol

Off-white solid (5.7 mg, 12% yield).

¹H NMR (DMSO-d₆) δ 7.90 (br s, 2H), 7.48-7.39 (m, 4H), 5.27 (s, 2H), 4.77-4.68 (br s, 1H), 4.67-4.54 (br s, 1H), 3.52-3.37 (m, 2H), 3.00 (s, 3H), 1.55-1.35 (m, 2H), 1.33-1.22 (m, 1H), 0.87 (d, 3H), 0.80 (d, 3H);

MS (ESI⁺) m/z 422 [M+H]⁺.

### EXAMPLE 7

### (2R)-2-[{2-Amino-5-[(3-chlorobenzyl)oxy][1,3]thiazolo[4,5-d]pyrimidin-7-yl}(methyl)amino]-4-methylpentan-1-ol

Obtained as an off-white solid (3.4 mg, 7% yield) by using a procedure analogous to the one described in Example 5, step (c), with the exception that lithium diisopropyl amide (LDA) was used as base (at −78° C.) instead of n-butyl lithium.

¹H NMR (DMSO-d₆) δ 7.91 (br s, 2H), 7.48-7.33 (m, 4H), 5.29 (s, 2H), 4.71 (t, 1H), 4.62 (br s, 1H), 3.52-3.36 (m, 2H), 3.00 (s, 3H), 1.56-1.35 (m, 2H), 1.33-1.21 (m, 1H), 0.86 (d, 3H), 0.79 (d, 3H);

MS (ESI⁺) m/z 422 [M+H]⁺.

### EXAMPLE 8

### (2R)-2-[{2-amino-5-[(2-methoxybenzyl)oxy][1,3]thiazolo[4,5-d]pyrimidin-7-yl}(methyl)amino]-4-methylpentan-1-ol

Obtained as an off-white solid (6.0 mg, 12% yield) by using a procedure analogous to the one described in Example 5, step (c), with the exception that LDA was used as base (at −78° C.) instead of n-butyl lithium.

¹H NMR (DMSO-d₆) δ 7.88 (br s, 2H), 7.39-7.26 (m, 2H), 7.06-6.99 (m, 1H), 6.97-6.90 (m, 1H), 5.26 (s, 2H), 4.71 (br s, 1H) overlapping with 4.66 (br s, 1H), 3.81 (s, 3H), 3.52-3.36 (m, 2H), 3.00 (s, 3H), 1.56-1.37 (m, 2H), 1.33-1.22 (m, 1H), 0.88 (d, 3H), 0.81 (d, 3H);

MS (ESI⁺) m/z 418 [M+H]⁺.

### EXAMPLE 9

### (2R)-2-[[2-Amino-5-(benzyloxy)[1,3]thiazolo[4,5-d]pyrimidin-7-yl](methyl)amino]-4-methylpentan-1-ol

Off-white solid (7.6 mg, 9% yield).

¹H NMR (DMSO-d₆) δ 7.89 (br s, 2H), 7.45-7.26 (m, 5H), 5.28 (s, 2H), 4.72 (br s, 1H) overlapping with 4.64 (br s, 1H), 3.52-3.36 (m, 2H), 3.00 (s, 3H), 1.56-1.37 (m, 2H), 1.33-1.24 (m, 1H), 0.88 (d, 3H), 0.82 (d, 3H);

MS (ESI) m/z 388 [M+H]³⁰ .

### EXAMPLE 10

### (2R)-[{2-Amino-5-[(4-bromo-2-fluorobenzyl)-(R_{S},S_{S})-sulfinyl][1,3]thiazolo[4,5-d]pyrimidin-7-yl}(methyl)amino]-4-methylpentan-1-ol

### (a) (2R)-2-[(2-Amino-5-mercapto[1,3]thiazolo[4,5-d]pyrimidin-7-yl)(methyl)amino]-4-methylpentan-1-ol

A three-neck round bottomed flask was immersed in a dry ice/ethanol cooling bath and equipped with a dry ice/ethanol condenser. The system was flushed with nitrogen and ammonia (approximately 50 mL) was condensed into the flask. The product from Example 5, step (a) (1 g, 2.5 mmol) was added to the flask, resulting in a clear yellow solution. Small pieces of sodium metal (size 2-3 mm) was added one by one to the reaction mixture. When a persistent blue color (>20 sec) appeared, a spoon of solid NH₄Cl was added to quench the reaction. The ammonia was evaporated. Water (50 mL) was added and the mixture was neutralized with aq 1M HCl until pH 7. The precipitated yellow solid was collected by filtration, washed with water and dried in vacuo to yield 630 mg of the title compound (80% yield).

¹H NMR (DMSO-d₆) δ 12.78 (br s, 1H), 8.43 (br s, 2H), 4.84 (br, 2H), 3.52-3.38 (m, 2H), 3.01 (s, 3H), 1.55-1.33 (m, 2H), 1.32-1.20 (m, 1H), 0.87 (m, 6H);

MS (ESI⁺) m/z 314 [M+H]⁺.

### (b) (2R)-2-[{2-Amino-5-[(4-bromo-2-fluorobenzyl)thio][1,3]thiazolo[4,5-d]pyrimidin-7-yl}(methyl)amino]4-methylpentan-1-ol

The product from step (a) (300 mg, 0.96 mmol) and 4-bromo-2-fluorobenzyl bromide (257 mg, 0.96 mmol) were dissolved in DMSO (2.5 mL) under nitrogen. DIPEA(124 mg, 0.96 mmol) was added and the resulting solution was stirred at RT for 30 min. The reaction mixture was poured onto ice and the pale yellow precipitate was collected by filtration and washed with water. After drying in vacuo the crude product was purified by column chromatography on silica (CH₂Cl₂:EtOAc 70:30 to 30:70) resulting in 366 mg (76% yield) of the title compound as an off-white solid.

¹H NMR (DMSO-d₆) δ 8.00 (br s, 2H), 7.50 (m, 2H), 7.33 (dd, 1H), 4.73 (br s, 1H), 4.61 (br s, 1H), 4.30 (s, 2H), 3.50-3.35 (m, 2H), 2.98 (s, 3H), 1.53-1.33 (m, 2H), 1.29-1.20 (m, 1H), 0.85 (d, 3H), 0.79 (d, 3H)MS (ESI⁺) m/z 500, 502 [M+H]⁺.

### (c) (2R)-[{2-Amino-5-[(4-bromo-2-fluorobenzyl)-(R_{S},S_{S})-sulfinyl][1,3]thiazolo[4,5-d]pyrimidin-7-yl}(methyl)amino]-4-methylpentan-1-ol

The product from step (b) (50 mg, 0.10 mmol) was dissolved in MeOH (5 mL). Potassium to peroxymonosulfate (Oxone, 74 mg, 0.12 mmol) was added and the resulting inhomogeneous mixture was stirred at RT for 3 h. The reaction mixture was poured onto ice and the white precipitate was collected by filtration, washed with water and dried in vacuo. The crude product was purified by column chromatography on silica (CH₂Cl₂:EtOAc 40:60 to 0:100, followed by EtOAc:MeOH 95:5) resulting in 35 mg (68% yield) of the title compound as a white solid (1:1 mixture of two unresolved diastereoisomers).

¹H NMR (DMSO-d₆) δ 8.19 (br s, 2H), 7.48 (m, 1H), 7.33 (m, 1H), 7.13 (m, 1H), 4.78 (m, 1H), 4.67 (br s, 1H), 4.41 (d, 1H), 4.22 (d, 1H in one diastereomer), 4.19 (d, 1H in one diastereomer), 3.54-3.38 (m, 2H), 3.014 (s, 3H in one diastereomer) overlapping with 3.008 (s, 3H in one diastereomer), 1.55-1.15 (m, 3H), 0.85 (m, 6H);

MS (ESI⁺) m/z 516, 518 [M+H]⁺.

### EXAMPLE 11

### (2R)-2-[(2-Amino-5-{[2-(4-bromophenyl)ethyl}-(R_{S},S_{S})-sulfinyl}[1,3]thiazolo[4,5-d]pyrimidin-7-yl)amino]-4-methylpentan-1-ol

### (a) 1-Bromo-4-(2-bromoethyl)benzene

To a solution of 2-(4-bromophenyl)ethanol (1.2 g, 6.0 mmol) in CH₂Cl₂ (50 mL) at RT under nitrogen was added CBr₄ (1.98 g, 5.8 mmol) and PPh₃ (1.57 g, 5.8 mmol). After stirring at RT for 18 h the reaction mixture was concentrated and the residue diluted with Et₂O (30 mL) resulting in precipitation of triphenylphosphine oxide. The ethereal solution was decanted, evaporated and purified by flash chromatography (silica, hexane) to provide the title compound as a clear oil (59%).

¹H NMR (DMSO-d₆) δ 7.45 (d, 2 H), 7.15 (d, 2 H), 3.51 (t, 2 H), 3.17 (t, 2 H);

¹³C NMR (DMSO-d₆) δ 138.1, 133.4, 131.2, 122.5, 38.5, 27.2.

### (b) (2R)-2-[(2-Amino-5-{[2-(4-bromophenyl)ethyl]thio}[1,3]thiazolo[4,5-d]pyrimidin-7-yl)amino]-4-methylpentan-1-ol

The title compound was obtained as an off-white solid in 40% yield from the product of step (a) and (2R)-2-[(2-amino-5-mercapto[1,3]thiazolo[4,5-d]pyrimidin-7-yl)amino]-4-methylpentan-1-ol (WO 0276990 A1) by using the procedure described in Example 10, step (b), with the exception that the product was purified by preparative HPLC.

¹H-NMR (DMSO-d₆) δ 7.98 (s, 2H), 7.47 (d, 2H), 7.25 (d, 2H), 6.89 (d, 1H), 4.70 (t, 1H), 4.29 (br s, 1H), 3.45-3.28 (m, 2H, obscured by water peak), 3.24 (t, 2H), 2.94 (t, 2H), 1.62-1.57 (m, 1H), 1.46-1.34 (m, 2H), 0.86 (d, 3H), 0.82 (d, 3H);

MS (ESI⁺) m/z 482, 484 [M+H]⁺.

(c) (2R)-2-[(2-Amino-5-{[2-(4-bromophenyl)ethyl]-(R_{S},S_{S})-sulfinyl}[1,3thiazolo[4,5-d]pyrimidin-7-yl)amino]-4-methylpentan-1-ol

The title compound was obtained as a white solid (1:1 mixture of two unresolved diastereoisomers) from the product of step (b), by following the procedure described in Example 10, step (c) with the exceptions that the reaction was run at 5° C. and that the product was purified by preparative HPLC.

¹H-NMR (DMSO-d₆) δ 8.07 (s, 2H), 7.31 (d, 2H), 7.05 (t, 2H), 4.59 (br s, 1H), 4.15 (br s, 1H), 3.28-3.19 (m, 2H, obscured by water peak), 3.19-3.05 (m, 2H obscured by water peak), 2.89-2.82 (m, 2H), 2.79-2.73 (m, 1H), 2.67-2.62 (m, 1H), 1.49-1.44 (m, 1H), 1.33-1.24 (m, 2H), 0.75-0.67 (m, 6H);

MS (ESI+) m/z 498, 500 [M+H]⁺.

### EXAMPLE 12

### (2R)-2-[(2-Amino-5-{[2-(2-bromophenyl)ethyl]-(R_{S},S_{S})-sulfinyl}[1,3]thiazolo[4,5-d]pyrimidin-7-yl)amino]-4-methylpentan-1-ol

### (a) (2R)-2-[(2-Amino-5-{[2-(2-bromophenyl)ethyl]thio}[1,3]thiazolo[4,5-d]pyrimidin-7-yl)amino]-4-methylpentan-1-ol

The title compound was obtained as a white solid in 67% yield by following the procedure described in Example 11, step (b), but replacing 1-(2-bromoethyl)-3-chlorobenzene with 1-bromo-2-(2-bromoethyl)benzene (U.S. Pat. No. 6,284,796).

¹H-NMR (DMSO-d₆) δ 7.97 (s, 2H), 7.59 (dd, 1H), 7.41 (dd, 1H), 7.34 (dt, 1H), 7.18 (dt, 1H), 6.87 (d, 1H), 4.66 (t, 1H), 4.29 (br s, 1H), 3.42-3.30 (m, 2H), 3.27 (t, 2H), 3.09 (t, 2H), 1.67-1.54 (m, 1H), 1.47-1.32 (m, 2H), 0.85 (d, 3H), 0.83 (d, 3H);

MS (ESI⁺) m/z 482, 484 [M+H]⁺.

### (b) (2R)-2-[(2-Amino-5-{[2-(2-bromophenyl)ethyl]-(R_{S},S_{S})-sulfinyl}[1,3]thiazolo[4,5-d]pyrimidin-7-yl)amino}-4-methylpentan-1-ol

The title compound was obtained as a white solid (25% yield; 1:1 mixture of two unresolved diastereoisomers) from the product of step (a), by following the procedure described in Example 11, step (c).

¹H-NMR (DMSO-d₆) δ 8.20 (s, 2H), 7.56 (d, 1H), 7.35-7.26 (m, 3H), 7.16 (dt, 1H), 4.70 (unresolved t, 1H), 4.28 (br s, 1H), 3.47-3.29 (m, 2H), 3.28 (m, 2H in one diastereomer, obscured by the water peak), 3.22-3.08 (m, 2H), 2.91-2.83 (m, 2H in one diastereomer), 1.64-1.54 (m, 1H), 1.49-1.30 (m, 2H), 0.85 (t, 6H, in one diastereomer), 0.84 (d, 3H in one diastereomer), 0.79 (d, 3H in one diastereomer);

MS (ESI⁺) m/z 498, 500 [M+H]⁺.

### EXAMPLE 13

### (R)-2-[(2-Amino-5-{[2-(2-bromophenyl)ethyl]-(R_{S},S_{S})-sulfinyl}[1,3]thiazolo[4,5-d]pyrimidin-7-yl)(methyl)amino]-4-methylpentan-1-ol

### (a) (2R)-2-[(2-Amino-5-{[2-(2-bromophenyl)ethyl]thio}[1,3]thiazolo[4,5-d]pyrimidin-7-yl)(methyl)amino]-4-methylpentan-1-ol

The title compound was obtained as a solid in 66% yield from the product of Example 10, step (a), by following the procedure described in Example 11, step (b), but replacing 1-(2-bromoethyl)-3-chlorobenzene with 1-bromo-2-(2-bromoethyl)benzene (see Example 12, step (a)).

¹H-NMR (DMSO-d₆) δ 7.97 (s, 2H), 7.60 (dd, 1H), 7.41 (dd, 1H), 7.37 (dt, 1H), 7.18 (dt, 1H), 4.75 (t, 1H), 4.68 (br s, 1H), 3.28 (t, obscured by the water peak, 2H), 3.09 (t, 2H), 3.02 (s, 3H), 1.57-1.42 (m, 2H),1.32-1.22 (m, 1H), 0.87 (d, 3H), 0.82 (d, 3H);

MS (ESI⁺) m/z 496, 498 [M+H]⁺.

### (b) (R)-2-[(2-Amino-5-{[2-(2-bromophenyl)ethyl]-(R_{S},S_{S})-sulfinyl}[1,3]thiazolo[4,5-d]pyrimidin-7-yl)(methyl)amino]-4-methylpentan-1-ol

The title compound was obtained as a clear film (40% yield; 1:1 mixture of two unresolved diastereoisomers) from the product of step (a), by following the procedure described in Example 11, step (c).

¹H-NMR (CD₃OD) δ 7.50 (app d, 1H), 7.29 (app d, 1H), 7.32 (app t, 1H), 7.09 (app t, 1H), 4.84 (obscured by the water peak, 3H), 4.56 (br s, 1H), 3.65-3.57 (m, 2H), 3.55-3.34 (m, 2H), 3.30 (s, 3H), 3.28-3.20 (m, 2H in one diastereomer, obscured by the MeOH peak), 3.06-2.93 (m, 2H in one diastereomer), 1.62-1.42 (m, 2H), 1.36-1.24 (m, 1H), 0.95-0.85 (m, 6H);

MS (ESI⁺) m/z 512, 514 [M+H]⁺.

### EXAMPLE 14

### 2-[(2,3-Difluorobenzyl)oxy]-4-{[(1R)-1-(hydroxymethyl)-3-methylbutyl]amino}pteridin-7(8H)-one

### (a) 2-[(2,3-Difluorobenzyl)sulfonyl]-4-{[(1R)-1-(hydroxymethyl)-3-methylbutyl]amino}pteridin-7(8H)-one

2-[(2,3-Difluorobenzyl)thio]-4-{[(1R)-1-(hydroxymethyl)-3-methylbutyl]amino}pteridin-7(8H)-one (WO 01/062758) (1.0 g, 2.37 mmol) was dissolved in CH₃CN (120 mL) and water (80 mL). Potassium peroxymonosulfate (Oxone, 3.38 g, 5.50 mmol) was added and the resulting slurry was stirred at RT for 16 h. Na₂S₂O₃ solution was added and the CH₃CN was evaporated in vacuo. The residue was poured onto ice and the precipitate was collected by filtration, washed with water and dried in vacuo at 40° C. overnight resulting in 891 mg (83%) of the title compound as an off-white solid.

¹H NMR (DMSO-d₆) δ 13.5-13.0 (br s, 1H), 8.05 (br s, 1H), 7.91 (s, 1H), 7.47 (app q, 1H), 7.30-7.18 (m, 2H), 4.98 (dd, 2H), 4.83 (t, 1H), 4.41-4.38 (m, 1H), 3.55-3.35 (m, 2H), 1.60-1.50 (m, 2H), 1.41-1.35 (m, 1H), 0.88 (d, 3H), 0.87 (d, 3H);

MS (ESI⁺) m/z 454 [M+H]⁺.

### (b) 2-[(2,3-Difluorobenzyl)oxy]-4-{[(1R)-1-(hydroxymethyl)-3-methylbutyl]amino}pteridin-7(8H)-one

Solid NaH (17 mg, 0.7 mmol, 7 eq.) was added to a stirred solution of 2,3-difluorobenzyl alcohol (0.10 g, 0.7 mmol, 7 eq.) in dry benzene (5 mL) at 0° C. The solution was allowed to reach RT over 15 min. The product from step (a) (45 mg, 0.1 mmol, 1 eq.) was added as a solid and the mixture was heated to reflux for 1 h. After cooling to RT, the reaction was quenched by addition of saturated aqueous NH₄Cl (1 mL). The mixture was partitioned between EtOAc (10 mL) and water (10 mL). The organic phase was separated, dried over Na₂SO₄ and evaporated. The oily residue was purified by preparative HPLC to give the title compound as an off-white solid (4.5 mg, 11% yield).

¹H NMR (CDCl₃) δ 9.80-9.20 (br s, 1H), 7.80 (s, 1H), 7.69-7.29 (m, 3H), 6.50 (m, 1H), 5.49 (s, 2H), 4.41 (m, 1H), 3.78 (dd, 1H), 3.64 (dd, 1H), 1.68-1.48 (m, 3H), 0.95 (d, 3H), 0.91 (d, 3H);

MS (ESI⁺) m/z 406 [M+H]⁺.

The compounds of Examples 15 to 22 were prepared using the general method of Example 14, step (b), but replacing 2,3-difluorobenzyl alcohol with the appropriate alcohol.

### EXAMPLE 15

### 4-{[(1R)-1-(Hydroxymethyl)-3-methylbutyl]amino}-2-[(3-methoxybenzyl)oxy]pteridin-7(8H)-one

Off-white solid (3.6 mg, 9% yield).

¹H NMR (CDCl₃) δ 9.90-9.24 (br s, 1H), 7.84 (s, 1H), 7.39-7.23 (m, 2H), 6.92-6.80 (m, 2H), 6.48 (m, 1H), 5.52 (s, 2H), 4.44 (m, 1H), 3.73 (dd, 1H), 3.51 (s, 3H), 3.48 (dd, 1H), 1.70-1.49 (3H), 0.96 (d, 3H), 0.91 (d, 3H);

MS (ESI⁺) m/z 400 [M+H]⁺.

### EXAMPLE 16

### 2-[(2-Chloro-3-methoxybenzyl)oxy]-4-{[(1R)-1-(hydroxymethyl)-3-methylbutyl]amino}pteridin-7(8H)-one

Off-white solid (3.9 mg, 9% yield).

¹H NMR (CDCl₃) δ 10.02-9.54 (br s, 1H), 7.85 (s, 1H), 7.30-7.06 (m, 3H), 6.46 (m, 1H), 5.50 (s, 2H), 4.43 (m, 1H), 3.73 (dd, 1H), 3.57 (s, 3H), 3.51 (dd, 1H), 1.76-1.43 (m, 3H), 0.96 (d, 3H), 0.93 (d, 3H);

MS (ESI⁺) m/z 434 [M+H]⁺.

### EXAMPLE 17

### 4-{[(1R)-1-(Hydroxymethyl)-3-methylbutyl]amino}-2-(2-phenylethoxy)pteridin-7(8H)-one

Off-white solid (6.5 mg, 17% yield).

¹H NMR (CDCl₃) δ 10.00-9.51 (br s, 1H), 7.82 (s, 1H), 7.32-7.11 (m, 5H), 6.45 (m, 1H), 4.82 (t, 2H), 4.43 (m, 1H), 3.73 (dd, 1H), 3.57-3.50(m, 3H), 1.79-1.43 (m, 3H), 0.94 (d, 3H), 0.89 (d, 3H);

MS (ESI⁺) m/z 384 [M+H]⁺.

### EXAMPLE 18

### 4-{[(1R)-1-(Hydroxymethyl)-3-methylbutyl]amino}-2-(2-phenoxyethoxy)pteridin-7(8H)-one

Off-white solid (10% yield).

¹H-NMR (CD₃OD) δ 7.78 (s, 1H), 7.25 (app t, 2H), 6.19 (app t, 3H), 4.71 (obscured by protons in the water peak, 3H), 4.70 (t, 2H), 4.45 (septet, 1H), 4.31 (t, 2H) 3.62 (d, 2H), 1.74-1.64 (m, 1H), 1.64-1.56 (m, 1H), 1.52-1.42 (m, 1H), 0.96 (d, 3H), 0.94 (m, 3H);

MS (ESI⁺) m/z 400 [M+H]⁺.

### EXAMPLE 19

### 2-[(2-Chlorobenzyl)oxy]-4-{[(1R)-1-(hydroxymethyl)-3-methylbutyl]amino}pteridin-7(8H)-one

Off-white solid (5.6 mg, 14% yield).

¹H NMR (CDCl₃) δ 8.5-8.0 (br s, 1H), 7.81 (s, 1H), 7.52-7.50 (m, 2H), 7.40-7.36 (m, 2H), 6.50 (d, 1H), 5.49 (app t, 2H), 4.45-4.40 (m, 1H), 3.78 (dd, 1H), 3.64(dd, 1H), 1.68-1.48 (m, 3H), 0.95 (d, 3H), 0.91 (d, 3H);

MS (ESP) m/z 404 [M+H]⁺.

### EXAMPLE 20

### 2-[(4-Chlorobenzyl)oxy]-4-{[(1R)-1-(hydroxymethyl)-3-methylbutyl]amino}pteridin-7(8H)-one

Off-white solid (1.2 mg, 3% yield).

¹H NMR (CDCl₃) δ 9.5-9.0 (br s, 1H), 7.83 (s, 1H), 7.38 (d, 2H), 7.33 (d, 2H), 6.50 (d, 1H), 5.35 (app t, 2H), 4.42-4.39 (m, 1H), 3.79 (dd, 1H), 3.66(dd, 1H), 1.70-1.47 (m, 3H), 0.97 (d, 3H), 0.93 (d, 3H);

MS (ESI⁺) m/z 404 [M+H]⁺.

### EXAMPLE 21

### 4-{[(1R)-1-(Hydroxymethyl)-3-methylbutyl]amino}-2-[(4-methylbenzyl)oxy]pteridin-7(8H)-one

Off-white solid (1.2 mg, 3% yield).

¹H NMR (CDCl₃) δ 10.0-8.75 (br s, 1H), 7.80 (s, H), 7.32 (d, 2H), 7.15 (d, 2H), 6.46 (d, 1H), 5.35 (app t, 2H), 4.44-4.40 (m, 1H), 3.79 (dd, 1H), 3.64 (dd, 1H), 2.34 (s, 3H), 1.70-1.48 (m, 3H), 0.96 (d, 3H), 0.93 (d, 3H);

MS (ESI+) m/z 384 [M+H]⁺.

### EXAMPLE 22

### 4-{[(1R)-1-(Hydroxymethyl)-3-methylbutyl]amino}-2-[(3-methylbenzyl)oxy]pteridin-7(8H)-one

Off-white solid (1.5 mg, 4% yield).

¹H NMR (CDCl₃) δ 10.5-9.0 (br s, H), 7.79 (s, 1H), 7.26-7.21 (m, 3H), 7.11-7.10 (m, 1H), 6.51 (m, 1H), 5.35 (app t, 2H), 4.44-4.42 (m, 1H), 3.81 (dd, 1H), 3.65 (dd, 1H) 2.33 (s, 3H), 1.71-1.44 (m, 3H), 0.96 (d, 3H), 0.93 (d, 3H);

MS (ESI⁺) m/z 384 [M+H]⁺.

### EXAMPLE 23

### 2-[(3-Chlorobenzyl)oxy]-4-{](1S,2S)-2-hydroxy-1-(hydroxymethyl)propyl]amino}-7-oxo-7,8-dihydropteridine-6-carboxamide

### (a) Methyl 4-amino-2-(benzylthio)-7-oxo-7,8-dihydropteridine-6-carboxylate

Sodium metal (2.3 g, 100 mmol) was dissolved in MeOH (450 mL) and 2-benzylthio-4,5,6-triaminopyrimidine (Berezovskii, Jurkewitsch, J. Gen. Chem. USSR (Engl. Transl.) 1962, 32, 1637) (4.6 g, 18 mmol) was added. The mixture was stirred at RT for 20 min, then dimethyl ketomalonate (10.6 g, 72.5 mmol) was added dropwise, and the mixture was stirred for another 4.5 h. Water (300 mL) was added, and the pH was adjusted to 5 by dropwise addition of conc. aqueous HCl. The precipitate formed was filtered off, washed with water and dried overnight in vacuo to give 4.46 g (70%) of the title compound.

¹H NMR (DMSO-d₆) δ 13.00 (s, 1H), 8.03 (br s, 1H), 7.85 (br s, 1H), 7.49-7.21 (m, 5H), 4.36 (s, 2H), 3.84 (s, 3H);

MS (ESI) m/z 344 [M+H]⁺.

### (b) Methyl 2-(benzylthio)-4-bromo-7-oxo-7,8-dihydropteridine-6-carboxylate

The product of step (a) (5.0 g, 14.6 mmol) was dissolved in a mixture of bromoform (100 mL) and DMF (100 mL). The resulting suspension was homogenized at 110° C. and is isoamyl nitrite (23 mL) was added dropwise over 10 min. After the addition was complete the mixture was cooled to RT in an ice bath and then evaporated in vacuo (oil pump). EtOAc was added to the residue, and the mixture was stirred for 2 h. The precipitate formed was filtered off, the EtOAc layer was evaporated and the resulting crude product was purified by flash cromatography (hexanes:EtOAc 1:1) to give 1.42 g (24%) of the title compound.

MS (ESI⁺) m/z 407, 409 [M+H]⁺.

### (c) Methyl 2-(benzylthio)-4-{[(1S,2S)-2-hydroxy-1-(hydroxymethyl)propyl]amino}-7-oxo-7,8-dihydropteridine-6-carboxylate

The product of step (b) (759 mg, 1.86 mmol) was dissolved in of 1-methyl-2-pyrrolidinone (NMP) (5 mL), and N-ethyl-N,N-diisopropylamine (DIPEA) (1.2 mL, 7.0 mmol) and D-threoninol (196 mg, 1.86 mmol) were added. The resulting mixture was stirred at 80° C. for 18 h. After addition of water (10 mL) the pH was adjusted to 5 by addition of HOAc. The precipitate formed was filtered off, washed with water and dried to give 739 mg (92%) of the title compound, which was used in the subsequent step without further purification.

MS (ESI⁺) m/z 432 [M+H]⁺.

### (d) 2-(Benzylthio)-4-{[(1S,2S)-2-hydroxy-1-(hydroxymethyl)propyl]amino}-7-oxo-7,8-dihydropteridine-6-carboxamide

The product of step (c) (1.0 g, 2.32 mmol) was dissolved in MeOH (40 mL), and ammonia gas was bubbled through the solution for 24 h. The reaction mixture was evaporated to give 0.92 g (95% yield) of the title compound, which was used in the subsequent step without further purification.

MS (ESI⁺) m/z 417 [M+H]⁺.

### (e) 2-(Benzylsulfonyl)-4-{[(1S,2S)-2-hydroxy-1-(hydroxymethyl)propyl]amino}-7-oxo-7,8-dihydropteridine-6-carboxamide

The product from step (d) (208 mg, 0.5 mmol) was dissolved in MeOH:water (3:1, 12 mL), and potassium peroxymonosulfate (Oxone, 768 mg, 1.1 mmol) was added. The reaction mixture was stirred for 12 h at RT. The MeOH was evaporated in vacuo without heating. Water (2 mL) was added to the residue, which was then left at 4° C. for 12 h. The precipitate formed was filtered off, washed with water and dried to give 504 mg (61% yield) of the title compound, which was used in the subsequent step without further purification.

MS (ESI⁺) nm/z 449 [M+H]⁺.

### (f) 2-[(3-Chlorobenzyl)oxy]-4-{[(1S,2S)-2-hydroxy-1-(hydroxymethyl)propyl]amino}-7-oxo-7,8-dihydropteridine-6-carboxamide

Toluene (150 μL) was added to NaH (168 mg, 7.0 mmol; 60% in oil, washed by hexanes), followed by addition of 3-chlorobenzyl alcohol (1.0 g, 7.0 mmol). The mixture was stirred at RT until no further gas evolution was observed (ca. 40 min). The product from step (e) (55.6 mg, 0.124 mmol) was added, and the resulting mixture was stirred at 60° C. for 2 h. Saturated aqueous NH₄Cl was added and the mixture was stirred for another 30 min at 60° C. After cooling to RT, the organic phase was separated and triturated with a mixture of Et₂O:hexanes (3:1). The precipitate formed was filtered off and purified by preparative HPLC (eluent CH₃CN/0.1M NH₄OAc 30:70 to 70:30) to give 5 mg (9%) of the title compound as an off-white solid.

¹H NMR (DMSO-d₆) δ 7.71 (br s, 1H), 7.60-7.30 (m, 4H), 5.42-5.28 (m, 2H), 4.97 (d, 1H), 4.82 (t, 1H), 4.13-3.98 (m, 2H), 3.65-3.50 (m, 2H), 1.06 (d, 3H);

MS (ESI⁺) m/z 435 [M+H]⁺.

### EXAMPLE 24

### 2-[(2,3-Difluorobenzyl)-(R_{S},S_{S})-sulfinyl]-4-(1R)-1-(hydroxymethyl)-3-methylbutyl]amino}pteridin-7(8H)-one

2-[(2,3-Difluorobenzyl)thio]-4-{[(1R)-1-(hydroxymethyl)-3-methylbutyl]amino}pteridin-7(8H)-one (WO 01/062758) (100 mg, 0.24 mmol) was dissolved in MeOH (18 mL), and water (6 mL) was added. Potassium peroxymonosulfate (Oxone, 150 mg, 0.25 mmol) was to added and the reaction was stirred at RT for 2 h. The reaction mixture was poured into water and extracted with EtOAc, dried (MgSO₄), filtered and concentrated in vacuo. Et₂O was added to the remains, and the yellow solid was filtered off. The crude solid was purified by preparative thin layer chromatography (10% MeOH in EtOAc) to give the title compound as a white solid (unresolved mixture of diastereomers 1:1; 11 mg, 11% yield).

¹H-NMR (DMSO-d₆) δ 13.16 (s, 1H in one diastereomer), 13.12 (s, 1H in one diastereomer), 8.17 (t, 1H), 8.034 (s, 1H in one diastereomer) 8.027 (s, 1H in one diastereomer), 7.44-7.33 (m, 1H), 7.19-7.05 (m, 1H), 7.01-6.92 (m, 1H), 4.85-4.78 (m, 1H), 4.60 (t, 2H in one diastereomer), 4.36 (br s, 1H), 4.33 (t, 2H in one diastereomer), 3.55-3.42 (m, 2H), 1.62-1.47 (m, 2H), 1.44-1.32 (m, 1H), 0.92-0.82 (m, 6H);

MS (ESI⁺) m/z 438 [M+H]⁺.

### EXAMPLE 25

### 5-(Benzyloxy)-7-{[(1R)-1-(hydroxymethyl)-3-methylbutyl]amino}[1,3]thiazolo[4,5-d]pyrimidin-2(3H)-one

### (a) (2R)-2-({2-Chloro-5-[(2,3-difluorobenzyl)thio[[1,3]thiazolo[4,5-d]pyrimidin-7-yl}amino)-4-methylpentan-1-ol

(2R)-2-({2-Amino-5-[(2,3-difluorobenzyl)thio][1,3]thiazolo[4,5-d]pyrimidin-7-yl}amino)-4-methylpentan-1-ol (WO 00/09511) (20.0 g, 47 mmol) was dissolved in conc. HCl (750 mL). CH₃CN (600 mL) and water (350 mL) were added and the mixture was cooled to 0° C. A solution of NaNO₂ (3.24 g, 94 mmol) in water (20 mL) was then added portionwise, and the mixture was stirred at 0° C. for 1.5 h. The yellow solid which had formed was collected by filtration, washed with water and dried to give 16.3 g (88%) of the title compound as a pale yellow solid.

¹H NMR (DMSO-d₆) δ 8.15 (d, 1H), 7.42-7.28 (m, 2H), 7.20-7.10 (m, 1H), 4.50 (b s, 1H), 4.46 (app t, 2H), 4.38-4.25 (m, 1H), 3.42 (app q, 2H), 1.67-1.54 (m, 1H), 1.53-1.42 (m, 1H), 1.41-1.32 (m, H), 0.88 (d, 3H), 0.83 (d, 3H);

MS (ESI⁺) m/z 445 [M+H]⁺.

### (b) (2R)-2-({5-[(2,3-Difluorobenzyl)thio]-2-methoxy[1,3]thiazolo[4,5-d]pyrimidin-7-yl}amino)-4-methylpentan-1-ol

The product from step (a) (10.75 g, 24.4 mmol) was dissolved in MeOH and solid potassium hydroxide (2.74 g, 48.8 mmol) was added. The mixture was heated to 55° C. for 1 h, cooled to RT and then neutralized with 2N HCl. MeOH was removed by evaporation in vacuo, water was added to the residue and the crude product was collected by filtration. Recrystallization from CH₃CN gave title compound (9.25 g; 88%) as a pale orange solid.

¹H NMR (DMSO-d₆) δ 7.60 (d, 1H), 7.40-7.28 (m, 2H), 7.20-7.10 (m, 1H), 4.74 (t, 1H), 4.44 (app q, 2H), 4.29 (b s, 1H), 4.16 (s, 3H), 3.9-3.35 (m, 2H, partially under the water peak), 1.65-1.52 (m, 1H), 1.50-1.32 (m, 2H), 0.87 (d, 3H), 0.82 (d, 3H);

MS (ESI⁺) m/z 441 [M+H]⁺.

### (c) 5-[(2,3-Difluorobenzyl)thio]-7-{[(1R)-1-(hydroxymethyl)-3-methylbutyl]amino}[1,3]thiazolo[4,5-d]pyrimidin-2(3H)-one

The product from step (b) (8.83 g, 20.0 mmol) was suspended in dioxane (300 mL). Conc. HCl (1.5 mL) and water (1 mL) were added and the mixture was heated to 50° C. for 15 h. Solvents were removed in vacuo and the residue was suspended in CH₃CN (300 mL). The off white solid was filtered off, washed with CH₃CN and dried to afford 7.92 g (90%) of the title compound.

¹H NMR (DMSO-d₆) δ 12.43 (br s, 1H), 7.45-7.27 (m, 3H), 7.20-7.08 (m, 1H), 4.46 (b s, 2H), 4.39 (1H, under the water peak), 4.26 (br s, 1H), 3.42-3.28 (m, 2H), 1.62-1.50 (m, 1H), 1.48-1.39 (m, H), 1.38-1.28 (m, 1H), 0.86 (d, 3H), 0.81 (d, 3H);

MS (ESI⁺) m/z 427 [M+H]⁺.

### (d) 5-[(2,3-Difluorobenzyl)sulfonyl]-7-{[(1R)-1-(hydroxymethyl)-3-methylbutyl]amino}[1,3]thiazolo[4,5-d]pyrimidin-2(3H)-one

The product from step (c) (2.0 g, 4.68 mmol) was dissolved in CH₃CN (240 mL) and water (160 mL). Potassium peroxymonosulfate (Oxone, 6.32 g, 10.30 mmol) was added and the resulting inhomogeneous mixture was stirred at RT for 24 h. Sodium thiosulphate solution was added and the CH₃CN was evaporated in vacuo. The residue was poured onto ice and the precipitate was collected by filtration, washed with water and dried in vacuo at 40° C. overnight resulting in 1.76 g (82%) of the title compound as an off-white solid.

¹H NMR (DMSO-d₆) δ 13.10 (br s, 1H), 7.52-7.40 (m, 2H) 7.28-7.18 (m, 2H), 5.0-4.85 (app t, 2H), 4.77 (br s, 1H), 4.29 (br s, 1H), 3.34 (br s, 2H), 1.65-1.51 (m, 1H), 1.50-1.31 (m, 2H), 0.88 (d, 3H), 0.85 (d, 3H);

MS (ESI⁺) m/z 459 [M+H]⁺.

### (e) 5-(Benzyloxy)-7-{[(1R)-1-(hydroxymethyl)-3-methylbutyl]amino}[1,3]thiazolo[4,5-dipyrimidin-2(3H)-one

Solid sodium hydride (17 mg, 0.7 mmol) was added to a stirred solution of benzyl alcohol (76 mg, 0.7 mmol) in dry benzene (5 mL) at 0° C. The solution was allowed to reach RT over 15 min. The product from step (d) (46 mg, 0.1 mmol) was added as a solid, and the mixture was heated to reflux for 1 h. After cooling to RT, the reaction was quenched by the addition of of saturated aqueous NH₄Cl (1 mL). The mixture was partitioned between THF (10 mL) and water (10 mL). The organic phase was separated, dried over Na₂SO₄ and evaporated in vacuo. The oily residue was purified by preparative HPLC, to give the title compound as a crystalline solid (6.0 mg, 16% yield).

¹H NMR (DMSO-d₆) δ 12.05 (br s, 1H), 7.52-7.24 (m, 5H), 5.95 (d, 1H), 5.02 (s, 2H), 4.77-4.29 (m, 2H), 3.38-3.30 (m, 2H), 1.63 (m, 1H), 1.50-1.32 (m, 2H), 0.89 (d, 3H), 0.83 (d, 3H);

MS (ESI⁺) m/z 375 [M+H]⁺.

The compounds of Examples 26 and 27 were prepared using the general method of Example 25, step (e), but replacing benzyl alcohol with the appropriate alcohol.

### EXAMPLE 26

### 7-{[(1R)-1-(Hydroxymethyl)-3-methylbutyl]amino}-5-[(3-methoxybenzyl)oxy][1,3]thiazolo[4,5-d]pyrimidin-2(3H)-one

Off-white solid (4.8 mg, 12% yield).

¹H NMR (DMSO-d₆) δ 12.75 (br s, 1H), 7.44-7.24 (m, 2H), 6.91 (s, 1H), 6.84 (d, 1H), 5.90 (d, 1H), 5.22 (s, 2H), 4.70-4.31 (m, 2H), 3.48 (s, 3H), 3.40-3.30 (m, 2H), 1.62 (m, 1H), 1.50-1.31 (2H), 0.88 (d, 3H), 0.83 (d, 3H);

MS (ESI⁺) m/z 405 [M+H]⁺.

### EXAMPLE 27

### 7-{[(1R)-1-(Hydroxymethyl)-3-methylbutyl]amino}-5-(2-phenylethoxy)[1,3]thiazolo[4,5-d]pyrimidin-2(3H)-one

Off-white solid (8.1 mg, 21% yield).

¹H NMR (DMSO-d₆) δ 12.87 (br s, 1H), 7.44-7.16 (m, 5H), 5.90 (d, 1H), 4.81 (t, 2H), 4.58-4.30 (m, 2H), 3.42-3.32 (m, 2H), 3.29 (t, 2H), 1.63 (m, 1H), 1.52-1.31 (m, 2H), 0.88 (d, 3H), 0.80 (d, 3H);

MS (ESI⁺) m/z 389 [M+H]⁺.

### EXAMPLE 28

### 5-(Benzyloxy)-7-{[(1R)-1-(hydroxymethyl)butyl]amino}[1,3]thiazolo[4,5-d]pyrimidin-2(3H)-one

### (a) (2R)-2-{[2-Amino-5-(benzylthio)[1,3]thiazolo[4,5-d]pyrimidin-7-yl]amino}pentan-1-ol

5-(Benzylthio)-7-chloro[1,3]thiazolo[4,5-d]pyrimidin-2-amine (WO 00/09511) (2.03 g, 6.57 mmol) was dissolved in 1-methyl-2-pyrrolidinone (NMP) (12 mL). N-Ethyl-N,N-diisopropylamine (DIPEA) (2.25 mL, 13.1 mmol) and 2-amino-(2R)-1-pentanol (1.19 g, 11.5 mmol) were added and the mixture was heated to 110° C. for 4 days. After cooling to RT, the mixture was poured into water (200 mL). The yellow solid was collected by filtration, washed with water and used for the next step without further purification (yield 80%).

MS (ESI⁺) m/z 376 [M+H]⁺.

### (b) (2R)-2-{[5-(Benzylthio)-2-chloro[1,3]thiazolo[4,5-d]pyrimidin-7-yl]amino}pentan-1-ol

The product from step (a) (2.46 g, 6.57 mmol) was dissolved in CH₃CN (70 mL). Sodium nitrite (1.36 g, 19.71 mmol) and conc. HCl (25 mL) were added at 0° C. and the reaction mixture was stirred at 0° C. for 3 h. The reaction mixture was diluted with water and extracted with EtOAc (3×60 mL), and the combined organic phases were dried, filtered and concentrated to give 2.59 g (quantitative yield) of the title compound as a yellow solid.

MS (ESI⁺) m/z 395 [M+H]⁺.

### (c) (2R)-2-{[5-(Benzylthio)-2-methoxy[1,3]thiazolo[4,5-d]pyrimidin-7-yl]amino}pentan-1-ol

The product from step (b) (2.59 g, 6.57 mmol) was dissolved in MeOH (80 mL). KOH (737 mg, 13.14 mmol) was added and the reaction mixture was stirred for 1.5 h at 50° C. After cooling to RT, the MeOH was removed under reduced pressure, the residue was diluted with brine and extracted with EtOAc (3×50 mL), and the combined organic phases were dried, filtered and concentrated to give 2.56 g (quantitative yield) of title compound as a yellow solid.

MS (ESI⁺) m/z 391 [M+H]⁺.

### (d) 5-(Benzylthio)-7-{[(1R)-1-(hydroxymethyl)butyl]amino}[1,3]thiazolo[4,5-d]pyrimidin-2(3H)-one

The product from step (c) (2.56 g, 6.57 mmol) was dissolved in dioxane (50 mL). Conc. HCl (544 μL, 6.57 mmol) was added and the reaction mixture was stirred for 4 h at 50° C. After cooling to RT, about half of the dioxane was removed under reduced pressure. The residue was diluted with brine, extracted with EtOAc (3×50 mL), and the combined organic phases were dried and concentrated to give 2.2 g (89%) of the title compound as a brown solid. It was used in the subsequent step without further purification.

MS (ESI⁺) m/z 377 [M+H]⁺.

### (e) 5-(Benzylsulfonyl)-7-{[(1R)-1-(hydroxymethyl)butyl]amino}[1,3]thiazolo[4,5-d]pyrimidin-2(3H) -one

The product from step (d) (1360 mg, 3.61 mmol) was dissolved in CH₃CN (85 mL) and water (56 mL). Potassium peroxymonosulfate (Oxone, 4 g, 6.51 mmol) was added and the resulting inhomogeneous mixture was stirred at RT for 24 h. The reaction mixture was concentrated to about one fifth of the original volume and extracted with EtOAc (3×40 mL). The combined organic phases were dried, filtered and concentrated to give 1.46 g (99%) of the title compound as a pale yellow powder.

MS (ESI⁺) m/z 409 [M+H]⁺.

### (f) 5-(Benzyloxy)-7-{[(1R)-1-(hydroxymethyl)butyl]amino}[1,3]thiazolo[4,5-d]pyrimidin-2(3H)-one

NaH (17 mg, 0.71 mmol) was added to a slurry of the product from step (e) (29 mg, 0.071 mmol) and benzyl alcohol (77 mg, 0.71 mmol) ) in dry benzene (0.5 mL) at RT. The reaction mixture was stirred for a few minutes at RT, and then heated to 40° C. for 50 min. After cooling to RT, the reaction mixture was quenched with water (0.1 mL) and concentrated. The residue was dissolved in DMSO (1 mL) and then purified by preparative HPLC to give 13.5 mg (52.7%) of the title compound as an off-white solid.

¹H NMR (DMSO-d₆) δ 12.25 (s, 1H), 7.44-7.29 (m, 5H), 5.29 (d, 1H), 5.25 (d, 1H), 4.65 (t, 1H), 4.13 (br s, 1H), 3.46-3.40 (m, 1H), 1.61-1.51 (m, 1H), 1.46-1.14 (m, 3H), 0.84 (t, 3H);

MS (ESI⁺) m/z 361 [M+H]⁺.

### EXAMPLE 29

### 7-{[(1R)-1-(Hydroxymethyl)butyl]amino}-5-{[(1S)-1-phenylethyl]oxy}[1,3]thiazolo[4,5-d]pyrimidin-2(3H)-one

The product from Example 28, step (e) (62 mg, 0.15 mmol) and (S)-1-phenylethanol (185 mg, 1.51 mmol) were dissolved in dry THF (2 mL) at RT, and n-BuLi (1.6M in hexanes, 0.85 mL, 1.36 mmol) was added. After stirring for 15 min at RT, the reaction mixture was heated to 50° C. for 24 h, cooled to RT and concentrated. The residue obtained was dissolved in DMSO (1 mL) and then purified by preparative HPLC to give 11.4 mg (20%) of the title compound as a slightly yellowish oil.

¹H NMR (CDCl₃) δ 7.41-7.23 (m, 5H), 5.90 (q, 1H), 4.60 (br d, 1H), 4.21-12 (m, 1H), 3.48 (dd, 1H), 3.42 (dd, 1H), 2.09 (s, 3H), 1.65-1.37 (m, 4), 0.97 (t, 3H);

MS (ESI⁺) m/z 375 [M+H]⁺.

### EXAMPLE 30

### N-(3-{[(7-{[(1R)-1-(Hydroxymethyl)butyl]amino}-2-oxo-2,3-dihydro[1,3]thiazolo[4,5-d]pyrimidin-5-yl)oxy]methyl}phenyl)-N-methylmethanesulfonamide

### (a) Methyl 3-[methyl(methylsulfonyl)amino]benzoate

Solid NaOMe (260 mg, 4.79 mmol) was added to a solution of methyl 3-[(methylsulfonyl)amino]benzoate (Laurence, C.; Berthelot, M.; Lucon, M.; Tsuno, Y. Spectrochim. Acta Part A 1982, 38, 791-796) (500 mg, 2.18 mmol) and MeI (0.4 mL, 6.42 mmol) in a mixture of THF (15 mL) and MeOH (15 mL). After 1 h at RT, the reaction mixture was heated to 50° C. for 1.5 h. The reaction mixture was cooled to RT, diluted with brine (30 mL) and extracted with EtOAc (2×30 mL). The combined organic phases were dried over MgSO₄, filtered, and concentrated, and the residue was purified by preparative HPLC to give 436 mg (82.2%) of the title compound as a white solid.

¹H NMR (CDCl₃) δ 8.00-7.91 (m, 2H), 7.60 (d, 1H), 7.44 (t, 1H), 3.89 (s, 3H), 3.33 (s, 3H), 2.83 (s, 3H);

MS (ESI⁺) m/z 244 [M+H]⁺.

### (b) N-[3-(Hydroxymethyl)phenyl]-N-methylmethanesulfonamide

Lithium borohydride (195 mg, 8.96 mmol) was added to a solution of the product from step (a) (436 mg, 1.79 mmol) in THF (25 mL). The reaction mixture was stirred for 2 h at RT, and then 20 h at 50° C. After cooling to RT, the mixture was diluted with brine (30 mL) and extracted with EtOAc (2×40 mL), dried over MgSO₄, and concentrated. The residue was purified by flash chromatography (0-5% MeOH in CHCl₃) to give 360 mg (93%) of the title compound as colourless oil.

¹H NMR (CDCl₃) δ 7.31-7.27 (m, 2H), 7.21-7.16 (m, 2H), 4.57 (s, 2H), 3.22 (s, 3H), 2.75 (s, 3H);

MS (ESI⁺) m/z 216 [M+H]⁺.

### (c) N-(3-{[(7-{[(1R)-1-(Hydroxymethyl)butyl]amino}-2-oxo-2,3-dihydro[1,3]thiazolo[4,5-d]pyrimidin-5-yl)oxy]methyl}phenyl)-N-methylmethanesulfonamide

n-BuLi (0.175 mL, 0.28 mmol, 1.6M in hexanes) was added to a stirred solution of N-[3-(hydroxymethyl)phenyl]-N-methyl-methanesulfonamide (from step (b), 60 mg, 0.28 mmol) and the product from Example 28, step (e) (36.5 mg, 0.089 mmol) in dry THF (1 mL). The resulting mixture was stirred at 50° C. for 18 h. After cooling to RT, the reaction mixture was concentrated, and the residue dissolved in DMSO (0.5 mL) and then purified by preparative HPLC to give 4 mg (9.6%) of the title compound as a white solid.

MS (ESI⁺) m/z 468 [M+H]⁺.

### EXAMPLE 31

### N-(3-{[(7-{[(1R)-1-(Hydroxymethyl)-2-methylpropyl]amino}-2-oxo-2,3-dihydro[1,3]thiazolo[4,5-d]pyrimidin-5-yl)oxy]methyl}phenyl)-methanesulfonamide

### (a) (2R)-2-{[5-(Benzylthio)-2-chloro[1,3]thiazolo[4,5-d]pyrimidin-7-yl]amino}-3-methylbutan-1-ol

A suspension of (2R)-2-{[2-amino-5-(benzylthio)[1,3]thiazolo[4,5-d]pyrimidin-7-yl]amino}-3-methylbutan-1-ol (WO 02/76990) (4.00 g, 10.7 mmol) in conc. HCl (150 mL) and CH₃CN (110 mL) was cooled to 0° C. Sodium nitrite (1.47 g, 21.3 mmol) was added and the solution was stirred at 0° C. for 1 h. Water (640 mL) was added and the resulting mixture was stirred for 15 min followed by filtration of the precipitate. The solid was washed with water and dried in vacuo over P₂O₅ at RT for 48 h resulting in 3.54 g (84%) of the title compound as a pink solid.

¹H NMR (DMSO-d₆) δ 8.10 (d, 1H), 7.44-7.39 (m, 2H), 7.32-7.26 (m, 2H), 7.25-7.19 (m, 1H), 4.81-4.49 (br s, 1H), 4.39 (d, 1H), 4.34 (d, 1H), 4.14-4.05 (m, 1H), 3.57-3.45 (m, 2H), 1.98-1.87 (m. 1H), 0.92-0.80 (m, 6H);

MS (ESI⁺) m/z 395 [M+H]⁺.

### (b) (2R)-2-{[5-(Benzylthio)-2-methoxy[1,3]thiazolo[4,5-d]pyrimidin-7-yl]amino}-3-methylbutan-1-ol

Using the product of step (a) as starting material, the title compound was obtained as a beige solid (67%) by following the general method described in Example 25, step (b).

¹H NMR (DMSO-d₆) δ 7.61-7.54 (m, 1H), 7.45-7.36 (m, 2H), 7.35-7.19 (m, 3H), 4.66-4.58 (m, 1H), 4.41-4.30 (m, 2H), 4.19-4.02 (m, 4H), 3.58-3.43 (m, 2H), 1.97-1.86 (m. 1H), 0.92-0.80 (m, 6H);

MS (ESI⁺) m/z 391 [M+H]⁺.

### (c) 5-(Benzylthio)-7-{[(1R)-1-(hydroxymethyl)-2-methylpropyl]amino}[1,3]thiazolo[4,5-d]pyrimidin-2(3H)-one

Using the product of step (b) as starting material, the title compound was obtained as a light orange solid (68%) by following the general method described in Example 25, step (c).

¹H NMR (DMSO-d₆) δ 12.36 (br s, 1H), 7.43-7.38 (m, 2H), 7.32-7.19 (m, 4H), 4.57 (app t, 1H), 4.33 (d, 1H), 4.28 (d, 1H), 4.08-3.97 (m, 1H), 3.54-3.41 (m, 2H), 1.93-1.83 (m. 1H), 0.87-0.79 (m, 6H);

MS (ESI⁺) m/z 377 [M+H]⁺.

### (d) 5-(Benzylsulfonyl)-7-{[(1R)-1-(hydroxymethyl)-2-methylpropyl]amino}[1,3]thiazolo[4,5-d]pyrimidin-2(3H)-one

Using the product of step (c) as starting material, the title compound was obtained as a pale yellow powder (99%) by following the general method described in Example 25, step (d).

MS (ESI⁺) m/z 409 [M+H]⁺.

### (e) N-(3-{[(7-{[(1R)-1-(Hydroxymethyl)-2-methylpropyl]amino}-2-oxo-2,3-dihydro[1,3]thiazolo[4,5-d]pyrimidin-5-yl)oxy]methyl}phenyl)methanesulfonamide

Solid sodium hydride (18 mg, 0.75 mmol) was added to a stirred solution of the product of step (d) (28.5 mg, 0.069 mmol) and N-[3-(hydroxymethyl)phenyl]-methanesulfonamide (WO 01/90070) (35 mg, 0.17 mmol) in a mixture of toluene (0.2 mL) and 1-methyl-2-pyrrolidinone (0.2 mL) at RT. The reaction mixture was stirred for 16 h at 50° C. After cooling to RT, the reaction mixture was quenched with water (0.1 mL) and concentrated. The residue was dissolved in DMSO (1 mL), and purified by preparative HPLC to give 4.5 mg (14.3%) of the title compound as an off-white solid.

MS (ESI⁺) m/z 454 [M+H]⁺.

### EXAMPLE 32

### 5-(Benzyloxy)-7-{[1-(hydroxymethyl)cyclopentyl]amino}-[1,3]thiazolo[4,5-d]pyrimidin-2(3H)-one

### a) (1-{[2-Amino-5-(benzylthio)[1,3]thiazolo[4,5-d]pyrimidin-7-yl]amino}cyclopentyl)methanol

The title compound was prepared using the general method of Example 28, step (a), but replacing 2-amino-(2R)-1-pentanol with cycloleucinol. The yellow solid was collected by filtration, washed with water and used for the next step without further purification.

MS (ESI⁺) m/z 388 [M+H]⁺.

### b) 5-(Benzylthio)-7-{[1-(hydroxymethyl)cyclopentyl]amino}[1,3]thiazolo[4,5-d]pyrimidin-2(3H)-one

The product from step (a) (1.2 g, 3.1 mmol) was suspended in water (150 mL), DMSO (10 mL) was added, and the mixture was heated to 80° C. Solid sodium nitrite (2.14 g, 31 mmol) was added in one portion and the mixture was heated at 80° C. for 3 h. After cooling to RT, acetic acid (10 mL) was added, and the white precipitate was collected by filtration. Purification of the crude product by flash column chromatography (EtOAc:CH₂Cl₂ 30:70) afforded the title compound (288 mg, 24% over two steps) as a white solid.

¹H NMR (DMSO-d₆) δ 12.45 (s, 1H), 7.44-7.22 (m, 5H), 7.0 (br s, 1H), 4.77 (t, 1H), 4.33 (s, 2H), 3.63 (d, 2H), 1.98 (m, 2H), 1.75 (m, 2H), 1.60 (m, 2H), 1.49 (m, 2H);

MS (ESI⁺) m/z 389 [M+H]⁺.

### c) 5-(Benzylsulfonyl)-7-{[1-(hydroxymethyl)cyclopentyl]amino}[1,3]thiazolo[4,5-d]pyrimidin-2(3H)-one

The title compound was prepared from the product of step (b), by following the procedure used in Example 25, step (d), and was obtained as an off-white solid in 86% yield.

¹H NMR (DMSO-d₆) δ 12.45 (s, 1H), 7.45-7.22 (m, 5H), 7.11 (br s, 1H), 4.93 (t, 1H), 4.82 (s, 2H), 3.60 (d, 2H), 1.98 (m, 2H), 1.72 (m, 2H), 1.60 (m, 2H), 1.46 (m, 2H);

MS (ESI⁺) m/z 421 [M+H]⁺.

### (d) 5-(Benzyloxy)-7-{[1-(hydroxymethyl)cyclopentyl]amino}[1,3]thiazolo[4,5-d]pyrimidin-2(3H)-one

Solid sodium hydride (17 mg, 0.7 mmol) was added to a stirred mixture of benzyl alcohol (ca. 850 μL) and toluene (ca. 150 μL) at 60° C. The solution was stirred at that temperature for 15 min, then the product of step (c) (42 mg, 0.1 mmol; 1 eq) was added as a solid in one portion, and the mixture was stirred at 60° C. for 1 h. After cooling to RT, the reaction was quenched by addition of saturated aqueous NH₄Cl (1 mL). The mixture was then partitioned between THF (10 mL) and water (10 mL). The organic phase was separated, dried, and concentrated. The residual oil was then triturated with EtOAc:hexane 1:1 (about 15 mL). The residue was purified by preparative HPLC to give the title compound as an off-white crystalline solid (16% yield).

¹H NMR (DMSO-d₆) δ 12.50 (s, 1H), 7.44-7.22 (m, 5H), 7.11 (br s, 1H), 5.13 (s, 2H), 4.90 (t, 1H), 3.71 (d, 2H), 1.98 (m, 2H), 1.72 (m, 2H), 1.60 (m, 2H), 1.45 (m, 2H);

MS (ESI⁺) m/z 373 [M+H]⁺.

The compounds of Examples 33 to 40 were prepared using the general method of Example 32, step (d), but replacing benzyl alcohol with the appropriate alcohol.

### EXAMPLE 33

### 7-{[1-(Hydroxymethyl)cyclopentyl]amino}-5-[(2-methylbenzyl)oxy][1,3]thiazolo[4,5-d]pyrimidin-2(3H)-one

Off-white solid (6.5 mg, 17% yield).

¹H NMR (DMSO-d₆) δ 12.55 (s, 1H), 7.40 (d, 1H), 7.35-7.22 (m, 3H), 7.08 (br s, 1H), 5.13 (s, 2H), 4.85 (t, 1H), 3.73 (d, 2H), 2.33 (s, 3H), 2.00 (m, 2H), 1.72 (m, 2H), 1.63 (m, 2H), 1.45 (m, 2H);

MS (ESI⁺) m/z 387 [M+H]⁺.

### EXAMPLE 34

### 7-{[1-(Hydroxymethyl)cyclopentyl]amino}-5-[(3-methylbenzyl)oxy][1,3]thiazolo[4,5-d]pyrimidin-2(3H)-one

Off-white solid (6.5 mg, 17% yield).

¹H NMR (DMSO-d₆) δ 12.52 (s, 1H), 7.37-7.20 (m, 3H), 7.05 (br s, 1H), 6.92 (d, 1H), 5.19 (s, 2H), 4.82 (t, 1H), 3.70 (d, 2H), 2.38 (s, 3H), 2.00 (m, 2H), 1.76 (m, 2H), 1.63 (m, 2H), 1.43 (m, 2H);

MS (ESI⁺) m/z 387 [M+H]⁺.

### EXAMPLE 35

### 5-[(2-chlorobenzyl)oxy]-7-{[1-(hydroxymethyl)cyclopentyl]amino}[1,3]thiazolo[4,5-d]pyrimidin-2(3H)-one

Off-white solid (5.7 mg, 14% yield).

¹H NMR (DMSO-d₆) δ 12.42 (s, 1H), 7.31-7.05 (m, 4H), 7.05 (br s, 1H), 5.09 (s, 2s), 4.80 (t, 1H), 3.70 (d, 2H), 1.93 (m, 2H), 1.75 (m, 2H), 1.60 (m, 2H), 1.43 (m, 2H);

MS (ESI⁺) m/z 406 [M+H]⁺.

### EXAMPLE 36

### 5-[(3-Chlorobenzyl)oxy]-7-{[1-(hydroxymethyl)cyclopentyl]amino}[1,3]thiazolo[4,5-d]pyrimidin-2(3H)-one

Off-white solid (6.1 mg, 15% yield).

¹H NMR (DMSO-d₆) δ 12.48 (s, 1H), 7.30 (s, 1H), 7.21-7.05 (m, 3H), 7.01 (br s, 1H), 5.12 (s, 2H), 4.81 (t, 1H), 3.75 (d, 2H), 1.97 (m, 2H), 1.75 (m, 2H), 1.63 (m, 2H), 1.41 (m, 2H);

MS (ESI⁺) m/z 406 [M+H]⁺.

### EXAMPLE 37

### 5-[(4-Chlorobenzyl)oxy]-7-{[1-(hydroxymethyl)cyclopentyl]amino}[1,3]thiazolo[4,5-d]pyrimidin-2(3H)-one

Off-white solid (6.1 mg, 15% yield).

¹H NMR (DMSO-d₆) δ 12.28 (s, 1H), 7.37 (d, 2H), 7.14 (d, 2H), 6.90 (br s, 1H), 5.22 (s, 2H), 4.88 (t, 1H), 3.75 (d, 2H), 1.99 (m, 2H), 1.75 (m, 2H), 1.64 (m, 2H), 1.40 (m, 2H);

MS (ESI⁺) m/z 406 [M+H]⁺.

### EXAMPLE 38

### 7-{[1-(Hydroxymethyl)cyclopentyl]amino}-5-[(2-methoxybenzyl)oxy][1,3]thiazolo[4,5-d]pyrimidin-2(3H)-one

Off-white solid (4.8 mg, 12% yield).

¹H NMR (DMSO-d₆) δ 12.17 (s, 1H), 7.34 (d, 1H), 7.20 (m, 1H), 6.97-6.89 (3H), 5.31 (s, 2H), 4.78 (t, 1H), 3.90 (s, 3H), 3.75 (d, 2H), 1.96 (m, 2H), 1.75 (m, 2H), 1.67 (m, 2H), 1.41 (m, 2H);

MS (ESI⁺) m/z 403 [M+H]⁺.

### EXAMPLE 39

### 7-{[1-(Hydroxymethyl)cyclopentyl]amino}-5-[(3-methoxybenzyl)oxy][1,3]thiazolo[4,5-d]pyrimidin-2(3H)-one

Off-white solid (7.2 mg, 18% yield).

¹H NMR (DMSO-d₆) δ 12.30 (s, 1H), 7.31-7.25 (m, 2H), 7.06 (d, 1H), 6.92 (s, 1H), 6.88 (br s, 1H), 5.31 (s, 2H), 4.78 (t, 1H), 3.95 (s, 3H), 3.70 (d, 2H), 1.99 (m, 2H), 1.75 (m, 2H), 1.63 (m, 2H), 1.40 (m, 2H);

MS (ESI⁺) m/z 403 [M+H]⁺.

### EXAMPLE 40

### 4-{[(7-{[1-(Hydroxymethyl)cyclopentyl]amino}-2-oxo-2,3-dihydro[1,3]thiazolo[4,5-d]pyrimidin-5-yl)oxy]methyl}benzonitrile

Off-white solid (5.2 mg, 13% yield).

¹H NMR (DMSO-d₆) δ 12.28 (s, 1H), 7.57 (d, 2H), 7.44 (d, 2H), 6.90 (br s, 1H), 5.37 (s, 2H), 4.80 (t, 1H), 3.75 (d, 2H), 2.02 (m, 2H), 1.73 (m, 2H), 1.60 (m, 2H), 1.40 (m, 2H);

MS (ESI⁺) m/z 398 [M+H]⁺.

### EXAMPLE 41

### (R,S)-7-[[1-(Hydroxymethyl)cyclopentyl]amino}-5-(1-phenylethoxy)-thiazolo[4,5-d]pyrimidin-2(3H)-one

n-BuLi (0.405 mL, 0.648 mmol, 1.6M in hexanes) was added to a stirred solution of racemic 1-phenyl-ethanol (87 mg, 0.72 mmol) in dry THF (0.2 mL) at RT. After 5 min stirring this mixture was added dropwise to the product of Example 32, step (c) (15.2 mg, 0.036 mmol) in dry THF (0.4 mL). When the addition was finished, the reaction mixture was stirred at 50° C. for 18 h. After cooling to RT, the reaction mixture was concentrated, and the residue dissolved in DMSO (1 mL) and then purified by preparative HPLC to give 3.3 mg (24%) of the title compound as a white solid.

MS (ESI⁺) m/z 387 [M+H]⁺.

### EXAMPLE 42

### 7-{[1-(Hydroxymethyl)cyclopentyl]amino}-5-{[(1S)-1-phenylethyl]oxy}1,3]thiazolo[4,5-d]pyrimidin-2(3H)-one

The title compound was prepared (7% yield) using the general method of Example 41, but replacing racemic 1-phenyl-ethanol with (1S)-1-phenyl-ethanol.

MS (ESI⁺) m/z 387 [M+H]⁺.

### EXAMPLE 43

### 5-{[2-(3-Chlorophenyl)-(R_{S},S_{S})-ethyl]sulfinyl}-7-{[(1R)-1-(hydroxymethyl)-3-methylbutyl]amino}[1,3]thiazolo[4,5-d]pyrimidin-2(3H)-one

### (a) (2R)-2-{2-Chloro-5-[2-chloro-7-((1R)-1-hydroxymethyl-3-methyl-butylamino)-thiazolo[4,5-d]pyrimidin-5-yldisulfanyl]-thiazolo[4,5-d]pyrimidin-7-ylamino}-4-methyl-pentan-1-ol

To a slurry of (2R)-2-[[2-amino-5-mercapto[1,3]thiazolo[4,5-d]pyrimidin-7-yl]amino]-methylpentan-1-ol (WO 02/76990) (7.50 g, 25 mmol) in a mixture of conc. HCl and CH₃CN (1:1, 300 mL) at 0° C. was added dropwise a solution of sodium nitrite (5.19 g, 75 mmol) in water (25 mL). The reaction mixture was stirred for 18 h at 0-5° C., and then poured onto ice (500 mL), and extracted with EtOAc with any remaining solid being filtered off. The combined organic phases were washed sequentially with saturated NaCl and saturated aqueous NaHCO₃ solution. The organic phase was dried and evaporated and the solid previously filtered off was added to this. The total solid was slurried in EtOAc which, after filtration, provided the title compound (6.3 g, 80%) as a pale yellow solid.

¹H NMR (400 MHz, DMSO-d₆; integrals are for the monomeric unit) δ 7.98 (d, 1H), 4.47 (t, 1 H), 3.99 (br s, 1 H), 3.19-3.14 (m, 2 H), 1.31-1.15 (m, 2 H), 1.02-0.94 (m, 1 H), 0.48 (d, 3 H), 0.30 (d, 3 H);

MS (ESI⁺) m/z 635 [M+H]⁺.

### (b) (2R)-2-{5-[7-((1R)-1-Hydroxymethyl-3-methyl-butylamino)-2-methoxy-thiazolo[4,5-d]pyrimidin-5-yldisulfanyl]-2-methoxy-thiazolo[4,5-d]pyrimidin-7-ylamino}-4-methyl-pentan-1-ol

To a solution of the product from step (a) (3.0 g, 4.7 mmol) in dry MeOH (200 mL) was added KOH (0.53 g, 9.4 mmol) dissolved in dry MeOH (5 mL). The reaction was maintained at 0-5° C. for 18 h. The solvent was evaporated and the residue taken up in MeOH/EtOAc (1: 1). This solution was rapidly chromatographed (silica, EtOAc) to provide the title compound (2.0 g, 68%) as a white solid.

MS (ESI⁺) m/z 627 [M+H]⁺.

### (c) 5-{[7-{[(1R)-1-(Hydroxymethyl)-3-methylbutyl]amino}-[1,3]thiazolo[4,5-d]pyrimidin-2(3H)-one-5-yldisulfanyl]-7-{[(1R)-1-(hydroxymethyl)-3-methylbutyl]amino}[1,3]thiazolo[4,5-d]pyrimidin-2(3H)-one

To a solution of the product from step (b) above (1.5 g, 2.4 mmol) in 1,4-dioxane (20 mL) was added a mixture of conc. HCl and water (40 mL, 1:1). The solution was then stirred at 45° C. for 18 h. The solvent was evaporated and the residue taken up in EtOAc (undissolved residue was filtered off and was found to be pure by LCMS). The solution was subjected to flash chromatography (silica, MeOH:EtOAc 5:95). The two samples were pooled together to give a white solid (600 mg, 42%, 75% pure by HPLC). This material was used without further purification in the ensuing reactions.

¹H NMR (DMSO-d₆; integrals are for the monomeric unit) δ 12.45 (s, 2H), 7.33 (d, 2H), 4.62 (t, 2H), 4.17 (br s, 2H), 1.48-1.31 (m, 4H), 1.25-1.14 (m, 2H), 0.72 (d, 6H), 0.56 (d, 6H);

MS (ESI⁺) m/z 599 [M+H]⁺.

### (d) 1-(2-Bromoethyl)-3-chlorobenzene

To a solution of 2-(3-chlorophenyl)ethanol (1.06 g, 6.0 mmol) in CH₂Cl₂ (50 mL) at RT under nitrogen was added CBr₄ (1.98 g, 5.8 mmol) and PPh₃ (1.57 g, 5.8 mmol). After stirring at RT for 18 h the reaction mixture was concentrated and the residue diluted with Et₂O (30 mL) resulting in precipitation of triphenylphosphine oxide. The ethereal solution was decanted, evaporated and purified via flash chromatography (silica, hexane) to provide 2-(3-chloro)phenylethyl bromide as a clear oil (57%).

¹H NMR (400 MHz, DMSO-d₆) δ 7.39-7.22 (m, 3 H), 7.18-7.09 (m, 1 H), 3.63-3.51 (m, 2 H), 3.25-3.17 (m, 2 H);

¹³C NMR (100.6 MHz, DMSO-d₆) δ 141.2, 134.6, 130.7, 129.3, 127.6, 127.3.

### (e) 5-{[2-(3-Chlorophenyl)ethyl]thio}-7-{[(1R)-1-(hydroxymethyl)-3-methylbutyl]amino}[1,3]thiazolo[4,5-d]pyrimidin-2(3H)-one

To a stirred solution of the product from step (c) above (30.0 mg, 0.05 mmol) in DMSO (0.5 mL) at RT was added NaBH₄ (5.6 mg, 0.125 mmol). Once effervescence had ceased, the product from step (d) above was added (20 mg, 0.09 mmol). The reaction was complete after 18 h at RT. Purification was achieved using preparative HPLC to give a white solid (90%).

¹H NMR (400 MHz, DMSO-d₆) δ 7.38-7.02 (m, 5 H), 6.08 (br s, 1 H), 4.29 (br s, 1 H), 3.60 (dd, 1 H), 3.49 (dd, 1 H), 3.26-3.18 (m, 2 H), 2.92 (t, 2 H), 1.63-1.55 (m, 1 H), 1.46-1.31 (m, 2 H), 0.82 (d, 3 H), 0.81 (d, 3 H);

MS (ESI⁺) m/z 439 [M+H]⁺.

### (f) 5-{[2-(3-Chlorophenyl)ethyl]-(R_{S},S_{S})-sulfinyl}-7-{[(1R)-1-(hydroxymethyl)-3-methylbutyl]amino}[1,3]thiazolo[4,5-d]pyrimidin-2(3H)-one

To a stirred solution of the product from step (e) above (15 mg, 0.025 mmol) in MeOH (2 mL) at RT was added potassium peroxymonosulfate (Oxone, 20.5 mg, 0.033 mmol). After 1.5 h the reaction was quenched by addition of water and saturated aqueous Na₂S₂O₃. The aqueous phase was extracted with EtOAc, dried and evaporated. Purification was achieved using preparative HPLC to give the title compound as a white solid (mixture of two unresolved diastereoisomers, 1:1; 27%).

¹H NMR (400 MHz, DMSO-d₆) δ 7.19-7.01 (m, 8 H), 6.99-6.98 (m, 2 H), 4.52 (t, 2 H), 4.07 (br s, 2 H), 2.87-2.80 (m, 2 H), 2.76-2.63 (m, 2 H), 1.27-1.20 (m, 2 H), 1.19-1.04 (m, 4 H), 0.70-0.66 (12 H, m);

MS (ESI⁺) m/z 455 [M+H]⁺.

### EXAMPLE 44

### 5-{[2-(2-Bromophenyl)ethyl]-(R_{S},S_{S})-sulfinyl}-7-{[(1R)-1-(hydroxymethyl)-3-methylbutyl]amino}[1,3]thiazolo[4,5-d]pyrimidin-2(3H)-one

### (a) 5-{[2-(2-Bromophenyl)ethyl]thio}-7-{[(1R)-1-(hydroxymethyl)-3-methylbutyl]amino}[1,3]thiazolo[4,5-d]pyrimidin-2(3H)-one

By following the procedure in Example 43, step (e), the title compound was obtained as a white solid in 58% yield from the reaction of the product of Example 43, step (c) with 1-(2-bromoethyl)-2-bromobenzene which, in turn, was prepared from 2-(2-bromophenyl)ethanol according to the procedure described in Example 43, step (d).

¹H-NMR (CDCl₃) δ 7.55 (unresolved dd, 1H), 7.29 (dd, 1H), 7.25 (unresolved dt, 1H), 7.10 (dt, 1H), 5.1 (br s, 1H), 3.82 (dd, 1H), 3.67 (dd, 1H), 3.89 (app dt, 2H), 3.16 (t, 2H), 1.85-1.63 (m, 1H), 1.58-1.42 (m, 2H), 0.95 (d, 3H), 0.93 (d, 3H);

MS (ESI⁺) m/z 483, 485 [M+H]⁺.

### (b) 5-{[2-(2-Bromophenyl)ethyl]-(R_{S},S_{S})-sulfinyl}-7-{[(1R)-1-(hydroxymethyl)-3-methylbutyl]amino}[1,3]thiazolo[4,5-d]pyrimidin-2(3H)-one

The title compound was obtained as a clear film in 62% yield (1:1 mixture of two unresolved diastereoisomers) from the product of step (a), by following the procedure described in Example 43, step (f).

¹H-NMR (CD₃OD) δ 7.50 (app dd, 1H), 7.27-7.21 (m, 2H), 7.12-7.06 (m, 1H), 4.97 (protons in the water peak, 3H) 4.45 (br s, 1H), 3.57-3.48 (m, 2H), 3.48-3.42 (m, 2H from one diastereomer), 3.37-3.30 (m, 2H from one diastereomer), 3.23-3.19 (m, 2H from one diastereomer), 3.07-3.01 (m, 2H from one diastereomer), 1.68-1.63 (m, 1H), 1.55-1.37 (m, 2H), 0.92 (d, 3H from one diastereomer), 0.90 (t, 6H from one diastereomer), 0.87 (d, 3H from one diastereomer);

MS (ESI⁺) m/z 499, 501 [M+H]⁺.

### EXAMPLE 45

### 5-[(2,3-Difluorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one

The title compound was obtained as a white solid in 41% yield (1:1 mixture of two unresolved diastereoisomers) starting from the product of Example 25, step (c) by following the general procedure described in Example 43, step (f).

¹H NMR (DMSO-d₆) δ 12.86 (b s, 1H), 7.68 (b s, 1H) 7.45-7.32 (m, 1H), 7.20-7.10 (m, 1H), 7.05-6.90 (m 1H), 4.77 (b s, 1H), 4.62-4.48 (app t, 1H), 4.38-4.20 (m, 2H), 3.90 (2H, partially under the water peak), 1.58 (b s, 1H), 1.50-1.30 (m, 2H), 0.88 (d, 3H), 0.84 (d, 3H);

MS (ESI⁺) m/z 443 [M+H]⁺.

### EXAMPLE 46

### 5-[Benzyl-(R_{S},S_{S})-sulfinyl]-7-{[(1R)-1-(hydroxymethyl)-3-methylbutyl]amino}[1,3]thiazolo[4,5-d]pyrimidin-2(3H)-one

### (a) (2R)-2-{[5-(Benzylthio)-2-methoxy[1,3]thiazolo[4,5-d]pyrimidin-7-yl]amino}-4-methylpentan-1-ol

To a suspension of (2R)-2-{[5-(benzylthio)-2-bromo[1,3]thiazolo[4,5-d]pyrimidin-7-yl]amino}-4-methylpentan-1-ol (1.90 g, 4.19 mmol) (WO 02/76990) in anhydrous MeOH (45 mL) was added potassium hydroxide (0.52 g, 9.22 mmol). The mixture was stirred at RT for 35 minutes followed by the addition of conc. HCl to pH 5. The solvent was evaporated and the crude solid was partitioned between water and methylene chloride. The organic phase was washed twice with water, brine, dried (MgSO₄), filtered, and the solvent was evaporated. The product was dried in vacuo at 35° C. for 2 h to give 1.72 g (quantitative yield) of the title compound as an orange solid.

¹H NMR (CDCl₃) δ 7.43 (d, 2H), 7.32-7.20 (m, 3H), 4.54 (d, 1H), 4.45-4.43 (m, 2H), 4.40-4.30 (m, 1H), 4.26 (s, 3H), 3.78-3.71 (m, 1H), 3.62-3.55 (m, 1H), 2.28 (t, 1H), 1.72-1.61 (m, 1H), 1.53-1.38 (m, 2H), 0.96-0.89 (m, 6H);

MS (ESI⁺) m/z 405 [M+H]⁺.

### (b) 5-(Benzylthio)-7-{[(1R)-1-(hydroxymethyl)-3-methylbutyl]amino}[1,3]thiazolo[4,5-d]pyrimidin-2(3H)-one

To a solution of the product from step (a) (1.72 g, 4.25 mmol) in 1,4-dioxane (50 mL) and water (1 mL) was added conc. HCl (0.91 mL). The mixture was heated at 45° C. for 15 h followed by evaporation of the solvent. A mixture of EtOAc/methylene chloride (5 mL, 30:70) was added and the solution was subjected to a stream of nitrogen gas for 2.5 h. The resulting solid was filtered off and washed with methylene chloride followed by EtOAc. The mother liquor was concentrated and flash chromatographed on silica (eluent EtOAc :methylene chloride 30:70). The two products were pooled resulting in 1.11 g (67% yield) of the title compound as a white solid.

¹H NMR (DMSO-d₆) δ 12.35 (br s, 1H), 7.43-7.39 (m, 2H), 7.31-7.19 (m, 4H), 4.36-4.23 (m, 3H), 3.45-3.28 (m, 1H) overlapping with H₂O-signal, 1.63-1.51 (m, 1H), 1.46-1.31 (m, 2H), 0.88-0.78 (m, 6H);

MS (ESI⁺) m/z 391 [M+H]⁺.

### (c) 5-[Benzyl-(R,S)-sulfinyl]-7-{[(1R)-1-(hydroxymethyl)-3-methylbutyl]amino}[1,3]thiazolo[4,5-d]pyrimidin-2(3H)-one

The title compound was obtained as a white solid in a 17% yield (1:1 mixture of two unresolved diastereoisomers) by following the method described in Example 43, step (f).

¹H NMR (DMSO-d₆) δ 12.82 (br s, 1H), 7.49 (br s, 1H), 7.34-7.26 (m, 3H), 7.17-7.10 (m, 2H), 4.76 (app t, 1H), 4.37 (dd, 2H from one diastereomer) overlapping with 4.30 (br s, 1H), 4.18 (dd, 2H from one diastereomer), 3.48-3.22 (m, 2H) overlapping with H₂O-signal, 1.59 (br s, 1H), 1.49-1.32 (m, 2H), 0.93-0.82 (m, 6H);

MS (ESI⁺) m/z 407 [M+H]⁺.

The compounds of Examples 47 to 49 were prepared using the general method of Example 43, step (f). The precursor sulfides were prepared according to the method of Example 43, step (e), but replacing 1-(2-bromoethyl)-3-chlorobenzene with the appropriate benzylic halide, all of which are commercially available.

### EXAMPLE 47

### 5-[(2-Chlorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one

### (a) 5-[(2-Chlorobenzyl)thiol-7-{[(1R)-1-(hydroxymethyl)-3-methylbutyl]amino}[1,3]thiazolo[4,5-d]pyrimidin-2(3H)-one

The title compound was obtained as a white solid in 52% yield from the product of Example 43, step (c), and 1-chloro-2-(chloromethyl)benzene.

¹H NMR (CD₃OD) δ 7.62-7.56 (m, 1H), 7.36-7.30 (m, 1H), 7.40-7.35 (m, 1H), 7.25-7.19 (m, 2H), 4.47 (dd, 2H) overlapping with 4.42 (br s, 1H), 3.56-3.47 (m, 2H), 1.71-1.59 (m, 1H), 1.56-1.46 (m, 1H), 1.45-1.36 (m, 1H), 0.92 (d, 3H), 0.89 (d, 3H);

MS (ESI⁺) m/z 425 [M+H]⁺.

### (b) 5-[(2-Chlorobenzyl)-(R_{S},S_{S})-sulfinyl]-7-{[(1R)-1-(hydroxymethyl)-3-methylbutyl]amino}[1,3]thiazolo[4,5-d pyrimidin-2(3H)-one

The title compound was obtained as an off-white solid in a 30% yield (1:1 mixture of two unresolved diastereoisomers).

¹H NMR (CD₃OD) δ 7.44 (d, 1H), 7.36-7.30 (m, 1H), 7.30-7.23 (m, 2H), 4.70 (dd, 2H from one diastereomer), 4.46 (br s, 1H), 4.37 (app t, 2H from one diastereomer), 3.58-3.47 (m, 2H), 1.72-1.60 (m, 1H), 1.55-1.38 (m,. 2H), 0.97-0.88 (m, 6H);

MS (ESI⁺) m/z 441 [M+H]⁺.

### EXAMPLE 48

### 5-[(4-Chlorobenzyl)-(R_{S},S_{S})-sulfinyl]-7-{[(1R)-1-(hydroxymethyl)-3-methylbutyl]amino}[1,3]thiazolo[4,5-d]pyrimidin-2(3H)-one

### (a) 5-[(4-Chlorobenzyl)thiol-7-{[(1R)-1-(hydroxymethyl)-3-methylbutyl]amino}[1,3]thiazolo[4,5-d]pyrimidin-2(3H)-one

The title compound was obtained as a white solid in 58% yield from the product of Example 43, step (c) and 1-chloro4(chloromethyl)benzene.

¹H NMR (CD₃OD) δ 7.41 (app d, 2H), 7.28 (app d, 2H), 4.39 (br s, 1H) overlapping with 4.34 (dd, 2H), 3.56-3.46 (m, 2H), 1.70-1.59 (m, 1H), 1.54-1.45 (m, 1H), 1.45-1.36 (m, 1H), 0.92 (d, 3H), 0.87 (d, 3H);

MS (ESI⁺) m/z 425 [M+H]⁺.

### (b) 5-[(4-Chlorobenzyl)-(R_{S},S_{S})-sulfinyl]-7-{[(1R)-1-(hydroxymethyl)-3-methylbutyl]amino}]1,3]thiazolo[4,5-d]pyrimidin-2(3H)-one

The title compound was obtained as a white solid in 25% yield (1:1 mixture of two unresolved diastereoisomers).

¹H NMR (CD₃OD) δ 7.28 (app t, 2H), 7.12 (app d, 2H), 4.45 (br s, 1H) overlapping with 4.42 (dd, 2H from one diastereomer), 4.27 (dd, 2H from one diastereomer), 3.58-3.47 (m, 2H), 1.72-1.57 (m, 1H), 1.55-1.35 (m, 2H), 0.98-0.86 (m, 6H);

MS (ESI⁺) m/z 441 [M+H]⁺.

### EXAMPLE 49

### 5-[Benzyl-(R_{S}S_{S})-sulfinyl]-7-{[(1R)-1-(hydroxymethyl)-2-methylpropyl]amino}[1,3]thiazolo[4,5-d]pyrimidin-2(3H)-one

The title compound was obtained from the product of Example 31, step (c) as a white solid in a 17% yield (1:1 mixture of two unresolved diastereoisomers) by following the procedure of Example 43, step (f).

¹H NMR (DMSO-d₆) δ 12.73 (br s, 1H), 7.63 (br s, 1H), 7.32-7.26 (m, 3H), 7.18-7.12 (m, 2H), 4.67-4.62 (m, 1H), 4.37 (dd, 2H from one diastereomer), 4.20 (d, 2H from one diastereomer), 4.12-4.02 (m, 1H), 3.60-3.45 (m, 2H), 1.95-1.85 (m, 1H), 0.93-0.83 (m, 6H);

MS (ESI+) m/z 393 [M+H]⁺.

### Pharmacological Screens

Materials

Recombinant human fractalkine (hCX₃CL1) was purchased from PeproTech Inc., UK. Recombinant [¹²⁵I]-fractalkine (human), with a specific activity of 2200 Ci/mmol, was purchased from NEN® Life Science Products, Inc., UK. Fluo4-AM was purchased from Molecular Probes, US. All other chemicals were of analytical grade.

Expression of Human Fractalkine Receptor (hCX₃CR1)

The complete human CX3CR1 cDNA (GenBank accession number U20350) was extracted from human brain mRNA (Superscript, Life Technologies) and ligated into pCR-Blunt II TOPO vector (InVitrogen). The insert corresponding hCX3CR1 was isolated and further subcloned into pcDNA3.1zeo. Plasmid DNA was prepared using Plasmid Midi Kit (Qiagen). Using Superfect Transfection Reagent (Qiagen) according to the manufacture's is protocol the expression plasmid for hCX₃CR1 was then introduced into human embryonic kidney suspension (HEKS) 293 cell line containing a vector for stable expression of a chimeric G-protein Gα_{qi5}. A stable clone was generated utilizing zeocin (500 μg/ml) and hygromycin (100 μg/ml) selection. For further applications the cells were maintained in Dulbecco's modified Eagle's medium/Ham's nutrient mix F12 (DMEMF12) containing pyridoxine and supplemented with 10% (v/v) fetal bovine serum, 2 mM L-glutamine, 100 U/ml penicillin and 100 mg/ml streptomycin, 250 μg/ml zeocin and 100 μg/ml hygromycin.

Ligand Binding Assay

For the competition binding assay cells were harvested in buffer containing 10 mM Tris-HCl, pH 7.4, 5 mM ethylenediaminetetra-aceticacid (EDTA) and 0.1 mg/ml bacitracin (a protease inhibitor) and centrifuged at 300×g for 10 min. Cell pellets were then resuspended in harvesting buffer, pooled and homogenised using Dounce homogeniser. Cell membranes were centrifuged at 48000×g for 10 min and then resuspended in harvesting buffer using Ultra-Turrax T8 (IKA Labortechnik, Germany). Protein concentration was determined in microtiter plates as described by Harrington (1990, Anal. Biochem. 186, 285-287). Membrane aliquotes were stored at −70° C. Receptor expression was confirmed with [¹²⁵I]-fractalkine binding using whole cells. Competition binding assays were performed in 2 ml 96-deep-well plates (Beckman, Germany) in a total volume of 1000 μl/well. Each well contained 10 pM [¹²⁵I]-fractalkine and membrane equivalent to receptor concentration of 1 pM in assay buffer [50 mM Hepes-KOH, pH 7.4, 10 mM MgCl₂, 1 mM EDTA, 0.1% (w/v) gelatin]. Test compounds were pre-dissolved in DMSO and added to reach a final concentration of 1% (v/v) DMSO. The assay was initiated with the addition of membranes and incubated at 25° C. for 24 h. Assay plates were filtrated with a Tomtec cell harvester (Tomtec, US) using ice-cold wash buffer (10 mM Hepes-KOH pH 7.4, 500 mM NaCl) and harvested onto printed filtermat B, GF/B (PerkinElmer LifeScience,US) presoaked in 0.3% polyetyhlenimine. MeltiLex solid scintillator (PerkinElmer LifeSciences,US) were melted onto filters and radioactivity was measured in a Wallac1205 Betaplate counter (PerkinElmer LifeScience, US).

### Solubility Assay

Method Description

100 μM Solutions in duplicate, prepared by dilution from a 10 mM DMSO stock solution of the test compound, were incubated in 0.1M phosphate buffer, pH 7.4, in a 96-well plate (PP plate, 350 μl U-shaped wells, COSTAR) on a plate bed shaker (IKA®-Schuttler MTS-4, IKA Labortechnik) at 300 rpm and room temperature (20-22° C.) for 24 hours.

The solutions were transferred to a MultiScreen™-R4 96-well filtration plate (LCR membrane, 0.4 μm hydrophilic PTFE, non-sterile glass-filled PP plate, 350 μl wells, Millipore) and filtered under vacuum to a 96-well collection plate (PP plate, 350 μl U-shaped wells, COSTAR), called the analyte plate, using Millipore Vacuum Manifold equipment. The analyte plate was covered by heat-sealing with an aluminium foil coated with a PP seal layer (AB-0813, pierceable sealing foil strong, ABgene).

LC-UV-MS analysis was performed using a generic LC method.

Single point quantification was performed against two 100 μM standards of the test compound dissolved in DMSO at the wavelength showing maximum UV absorbance as extracted from the DAD-trace (210-400 nm). The upper limit of the screen method is 100 μM with a LOQ of 0.1 μM.

Results

When tested in the ligand binding assay, the compounds of Examples 1 to 49 gave Kᵢ values of less than 10 μM, indicating that they are expected to show useful therapeutic activity. For example, the particular compounds of Examples 25 and 45 gave Kᵢ values of 44.6 and 38.0 nM respectively.

Representative solubility data are shown in the following Tables in which eight Examples from the present application are compared with the corresponding sulphide derivatives (X═S) from within the generic scope of WO 00/09511, WO 01/58907, WO 01/25242 and WO 01/62758:

| | | Solubility |
|---|---|---|
| Compound | | (μM) |
| 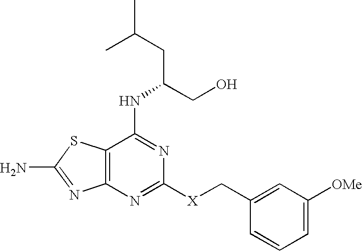 | X = O Example 2 X = S | 72.9 0.5 |
| 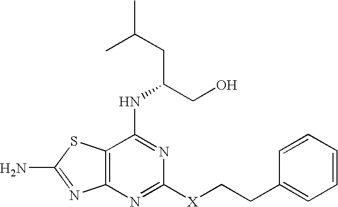 | X = O Example 3 X = S | 63.6 0.3 |
| 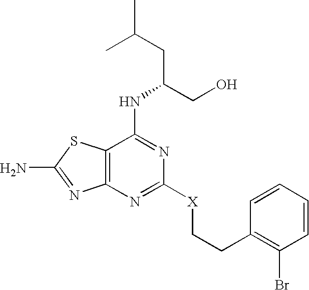 | X = S(O) Example 13 X = S | 33.8 0.0 |
| 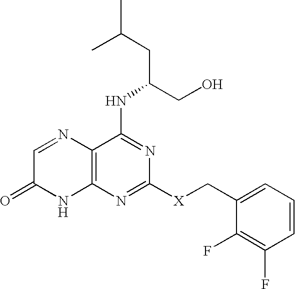 | X = S(O) Example 24 X = S | 44.0 1.3 |
| 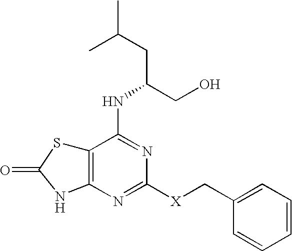 | X = O Example 25 X = S | 29 1.3 |
| 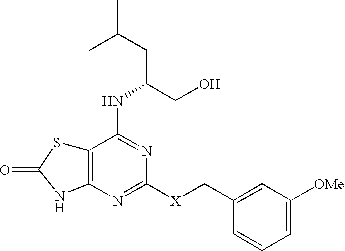 | X = O Example 26 X = S | 78.5 3.5 |
| 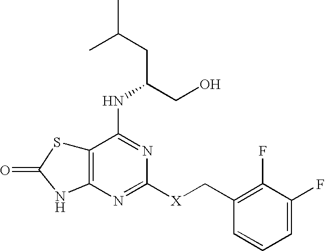 | X = S(O) Example 45 X = S | >100 2.1 |
| 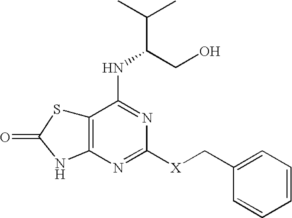 | X = S(O) Example 49 X = S | >100 8.5 |

1-10. (canceled)

11. A compound of formula (I) 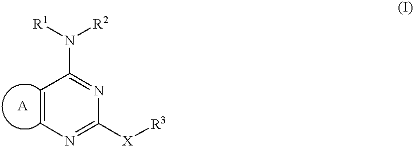
wherein:
A represents a group of formula (a) or (b) or (c): 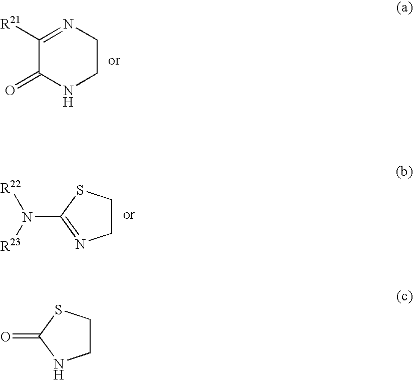
R¹ and R² independently represent H, C1 to 8 alkyl, C2 to 8 alkenyl, C2 to 8 alkynyl or C3 to 7 saturated or partially unsaturated cycloalkyl; the latter four groups being optionally further substituted by one or more groups selected independently from OH, C1 to 6 alkoxy, CH₂OR⁴, NR⁵R⁶, CO₂R⁷ and CONR⁸R⁹;
R³ represents C1 to 6 alkyl, C2 to 6 alkenyl, C2 to 6 alkynyl or C3 to 7 saturated or partially unsaturated cycloalkyl; said alkyl, alkenyl or alkynyl chain optionally including a O, NR¹⁰ or S atom in the chain; said alkyl, alkenyl, alkynyl or cycloalkyl group being optionally substituted by phenyl or a 5 or 6 membered heteroaromatic ring containing 1 to 3 heteroatoms selected independently from O, S and N; said phenyl or heteroaromatic ring being optionally further substituted by one or more groups selected independently from halogen, C1 to 4 alkyl, OH, C1 to 4 alkoxy, CN, CO2R¹¹, NR¹²R¹³, CONR¹⁴R¹⁵, SO₂R¹⁶, NR SO₂R¹⁸ and SO₂NR¹⁹R²⁰;
X represents O or S(O);
R²¹ represents H, CH₂OR²⁴, CH₂NR²⁴R²⁵, CO₂R²⁴ or CONR²⁴R²⁵;
R²² and R²³ independently represent H, C1 to 6 alkyl, C2 to 6 alkenyl or C3 to 7 saturated or partially unsaturated cycloalkyl; said alkyl, alkenyl or cycloalkyl group being optionally substituted by OR²⁴, NR²⁴R²⁵, CO₂R²⁵ or CONR²⁴R²⁵; or the group-NR²²R²³ together represents a 3 to 7 membered saturated azacyclic ring optionally incorporating one further heteroatom selected from O, S(O)ₙ and NR²⁶; and optionally substituted by OR²⁴; NR²⁴R²⁵, CO₂R²⁴ or CONR²⁴R²⁵;
n represents an integer 0, 1 or 2;
R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²⁴, R²⁵ and R²⁶ independently represent H or C1 to 6 alkyl;
and pharmaceutically acceptable salts thereof.

12. A compound according to claim 11 wherein R¹ represents H or CH₃.

13. A compound according to claim 11 wherein R represents C1 to 8 alkyl substituted by OH or C3 to 7 cycloalkyl substituted by OH or CH₂OR⁴.

14. A compound according to claim 11 wherein R³ represents C1 to 2 alkyl substituted by phenyl; said phenyl being optionally substituted by halogen, C1 to 6 alkoxy or CN.

15. A compound of formula (I), according to claim 11 or a pharmaceutically acceptable salt thereof, for use as a medicament.

16. A pharmaceutical formulation comprising a compound of formula (I), as defined in claim 11 or a pharmaceutically acceptable salt thereof, optionally in admixture with a pharmaceutically acceptable diluent or carrier.

17. A method of treating, or reducing the risk of, a human disease or condition in which antagonism of the CX₃CR1 receptor is beneficial which comprises administering to a person suffering from or susceptible to such a disease or condition, a therapeutically effective amount of a compound of formula (I), as defined in claim 11 or a pharmaceutically acceptable salt thereof.

18. The use of a compound of formula (I) as defined in claim 11 or a pharmaceutically acceptable salt thereof, in the manufacture of a medicament for the treatment or prophylaxis of human diseases or conditions in which antagonism of the CX₃CR1 receptor is beneficial.

19. The use of a compound of formula (I) as defined in claim 11 or a pharmaceutically acceptable salt thereof, in the manufacture of a medicament for the treatment or prophylaxis of neurodegenerative disorders, demyelinating disease, atherosclerosis or pain.

20. A process for the preparation of a compound of formula (I), as defined in claim 11 or a pharmaceutically acceptable salt thereof, wherein the process comprises:
(a) when X in formula (I) represents O, reaction of a compound of formula (II) 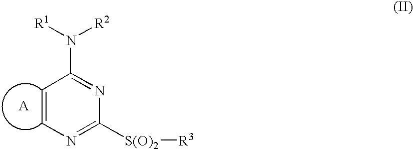
wherein A, R¹, R² and R³ are as defined in claim 11;
with a compound of formula (III)
R³—OH (III)
wherein R³ is as defined in claim 11 and is independent of the R³ group in formula (II); or
(b) when X in formula (I) represents S(O), oxidation of a compound of formula (IV) 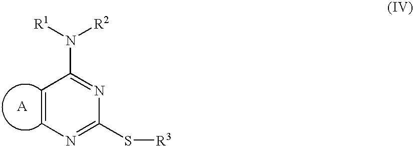
wherein A, R¹, R² and R³ are as defined in claim 11; with one equivalent of an oxidising agent;
and where necessary converting the resultant compound of formula (I), or another salt thereof, into a pharmaceutically acceptable salt thereof; or converting the resultant compound of formula (I) into a further compound of formula (I); and where desired converting the resultant compound of formula (I) into an optical isomer thereof.
